SEQUENCE LISTING (20 sequences)

SEQ ID NO: 1 - DNA, 4929 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcaccagtggtggcagcttcaggaaccggtttggtgctggtgctggaggcggctatggctttggaggtggtgccggtagtggatttggtttcggcggtggagctggtggtggctttgggcttggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagaccgtgaggcagaacctggagccgttgttcgagcagtacatcaacaacctcaggaggcagctggacagcatcgtgggggaacggggccgcctggactcagagctaagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggaccgaagccgagtcctggtatcagaccaagtatgaggagctgcagcagacagctggccggcatggcgatgacctccgcaacaccaagcatgagatctctgagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtggcagtggaagctactactccagcagcagtgggggtgtcggcctaagtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctagaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 2 - DNA, 4575 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccaccatgaccacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctatgggttggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaacgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaaagcttggtaccgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 3 - DNA, 6674 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccgcacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcacagaatgatgtgaaccctgtctctctgcagctggacctggacaggagattccagcttcaagaagtcatgatggagttccaggggcccatgcccgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactccctcagcccacagtgcaaccagttcacagggcagtgcccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgtaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcagcggacctgacaggactggagaagcgtgtggagcagatccgtgaccacatcaatgggcgcgtgctctactatgccacctgcaagtgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 4 - DNA, 11990 bp, Homo Sapiens
cgtgaggctccggtgcccgtcagtgggcagagcgcacatcgcccacagtccccgagaagttggggggaggggtcggcaattgaaccggtgcctagagaaagtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaggatccgatatcgtcgaccaccatgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgacccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagccaacctccgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccggctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacaaggcctccctggcccctctggtccccctggattaccgggaaagccaggcgaggatgggaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactgaattccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttacgcgtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaactagtccatggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcc

SEQ ID NO: 5 - DNA, 39 bp, Artificial Sequence
  Oligonucleotide KRT5_F
tttggatccaccatgtctcgccagtcaagtgtgtccttc

SEQ ID NO: 6 - DNA, 35 bp, Artificial Sequence
  Oligonucleotide KRT5_R
aataagcttctagctcttgaagctcttccgggagg

SEQ ID NO: 7 - DNA, 20 bp, Artificial Sequence
  Oligonucleotide KRT5_SF
cgaagccgagtcctggtatc

SEQ ID NO: 8 - DNA, 20 bp, Artificial Sequence
  Oligonucleotide KRT5_SR
ttggcgcactgtttcttgac

SEQ ID NO: 9 - DNA, 33 bp, Artificial Sequence
  Oligonucleotide KRT14_F
tttggatccaccatgaccacctgcagccgccag

SEQ ID NO: 10 - DNA, 34 bp, Artificial Sequence
  Oligonucleotide KRT14_R
aataagctttcagttcttggtgcgaaggacctgc

SEQ ID NO: 11 - DNA, 20 bp, Artificial Sequence
  Oligonucleotide KRT14_SF
tccaggagatgattggcagc

SEQ ID NO: 12 - DNA, 20 bp, Artificial Sequence
  Oligonucleotide KRT14_SR
ggatgactgcgatccagagg

SEQ ID NO: 13 - DNA, 34 bp, Artificial Sequence
  Oligonucleotide LAMB3_F
ttagtcgaccaccatgagaccattcttcctcttg

SEQ ID NO: 14 - DNA, 31 bp, Artificial Sequence
  Oligonucleotide LAMB3_R
atagaattcacttgcaggtggcatagtagag

SEQ ID NO: 15 - DNA, 20 bp, Artificial Sequence
  Oligonucleotide LAMB3_SF
cagaggagctgtttggggag

SEQ ID NO: 16 - DNA, 20 bp, Artificial Sequence
  Oligonucleotide LAMB3_SR
cccattgatgtggtcacgga

SEQ ID NO: 17 - DNA, 33 bp, Artificial Sequence
  Oligonucleotide COL7A1_F
atcgtcgaccaccatgacgctgcggcttctggt

SEQ ID NO: 18 - DNA, 29 bp, Artificial Sequence
  Oligonucleotide COL7A1_R
atagaattcagtcctgggcagtacctgtc

SEQ ID NO: 19 - DNA, 20 bp, Artificial Sequence
  Oligonucleotide COL7A1_SF
caaaggagagatgggggagc

SEQ ID NO: 20 - DNA, 20 bp, Artificial Sequence
  Oligonucleotide COL7A1_SR
atcatttccactggggcctg

(19) United States
(12) Patent Application Publication
(10) Pub. No.: US 2024/0307558 A1
(43) Pub. Date: Sep. 19, 2024

(54) Gene therapy DNA vector based on gene therapy DNA vector VTvaf17 carrying the therapeutic gene selected from the group of KRT5, KRT14, LAMB3, and COL7A1 genes for increasing the expression level of these therapeutic genes, method of its production and use, Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 carrying the gene therapy DNA vector, m

(71) Applicants: CELL AND GENE THERAPY LTD, London (GB); OBSCHESTVO S OGRANICHENNOI OTVETSTVENNOSTJU "PRORYVNYE INNOVATSIONNYE TEKHNOLOGII", Moscow (RU)

(72) Inventor: Natalia SAVELIEVA, Wienna (AT)

(21) Appl. No.: 18/269,582

(22) PCT Filed: Dec. 20, 2019
(86) PCT No.: PCT/RU2019/000990

§ 371 Date: Jun. 26, 2023

(30) Foreign Application Priority Data
Dec. 26, 2018 (RU) 2018146742

(51) Int. Cl. A61K 48/00; C07K 14/47; C07K 14/78; C12N 15/85

(52) U.S. Cl. CPC A61K 48/0058; C07K 14/4741; C07K 14/78; C12N 15/85; C12N 2800/107

(57) ABSTRACT

The invention refers to genetic engineering and can be used in biotechnology, medicine, and agriculture for the manufacture of gene therapy products. Gene therapy DNA vector based on the gene therapy DNA vector VTvaf1V carrying the therapeutic gene selected from the group of KRT5, KRT14, LAMB 3, and COL7A1 genes was constructed in order to increase the expression level of this therapeutic gene in humans and animals, while gene therapy DNA vector VTvaf17-KRT5, or VTvaf17-KRT14, or VTvaf17-LAMB3, or VTvaf17-COL7A1 has the nucleotide sequence SEQ ID No. 1, or SEQ ID No. 2, or SEQ ID No. 3, or SEQ ID No. 4, respectively. The gene therapy DNA vector contains no nucleotide sequences of viral origin and no antibiotic resistance genes, which ensures its safe use for gene therapy in humans and animals. A method of obtaining the specified vector, the use of the vector, a strain of Escherichia coli carrying the specified vector, and a method of industrial production of the specified vector are also provided.

Gene therapy DNA vector based on gene therapy DNA vector VTvaf17 carrying the therapeutic gene selected from the group of KRT5, KRT14, LAMB3, and COL7A1 genes for increasing the expression level of these therapeutic genes, method of its production and use, Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 carrying the gene therapy DNA vector, method of production thereof, method of gene therapy DNA vector production on an industrial scale.

### FIELD OF THE INVENTION

The invention refers to genetic engineering and can be used in biotechnology, medicine, and agriculture for the manufacture of gene therapy products.

### BACKGROUND OF THE INVENTION

Gene therapy is an innovative approach in medicine aimed at treating inherited and acquired diseases by means of delivery of new genetic material into a patient's cells to compensate for or suppress the function of a mutant gene and/or treat a genetic disorder. The final product of gene expression may be an RNA molecule or a protein molecule. However, most physiological processes in the body are associated with the functional activity of protein molecules, while RNA molecules are either an intermediate product in the synthesis of proteins or perform regulatory functions. Thus, the objective of gene therapy in most cases is to inject the organism with genes that provide transcription and further translation of protein molecules encoded by these genes. Within the description of the invention, gene expression refers to the production of a protein molecule with amino acid sequence encoded by this gene.

KRT5, KRT14, LAMB3, and COL7A1 genes included in the group of genes play a key role in several processes in human and animal organisms. The correlations between low/insufficient concentrations of these proteins and different adverse human states in some cases confirmed by disturbances in normal gene expression encoding these proteins was demonstrated. Thus, the gene therapy increase of expression of a gene selected from the group of KRT5, KRT14, LAMB3, and COL7A1 genes has potential to correct various conditions in humans and animals.

The COL7A1 gene encodes type VII collagen. Three pro-α1(VII) chains twist together to form a triplex procollagen molecule. Procollagen molecules are secreted by the cell and processed by enzymes to remove extra protein segments from the ends. Once these molecules are processed, they arrange themselves into long, thin bundles of mature type VII collagen.

Mutations in the COL7A1 gene cause dystrophic epidermolysis bullosa. Blisters most commonly occur in areas of minor injuries, such as the extensor surfaces of elbows and back of the hands and feet. Healing results in scarring, superficial epidermal cysts and hyperpigmentation. Some patients have nail dystrophy. Extracutaneous manifestations, including injuries to the urinary and gastrointestinal tracts, outer eye membranes, chronic anemia, osteoporosis, and growth retardation frequently occur. Patients with epidermolysis bullosa are at a high risk of cancer, in particular, formation of aggressive squamous cell carcinomas. DEBRA International founded in 1978 in the UK studies and treats the epidermolysis bullosa worldwide. According to DEBRA International, one patient per 50-100 thousand people is born in the world.

Various studies are currently underway worldwide on the treatment of epidermolysis bullosa in three directions: gene therapy, recombinant protein therapy, and cell-based therapy (stem cell use). All of these types of treatment are at different development stages.

Gene therapy approaches to the treatment of epidermolysis bullosa include different experimental treatments. In several studies for the correction of COL7A1 gene mutations, ex vivo genome editing technologies (Mencía et al., 2018), microinjections of linear DNA molecules encoding the COL7A1 gene (Mecklenbeck et al., 2002), cDNA integration using integrase enzymes (Ortiz-Urda et al., 2003), intradermal injections of lentiviral vectors (Woodley et al., 2004), mutation repair technology based on TALEN nucleases (Osborn et al., 2013), as well as injection of autologous cells, i.e. modified fibroblasts or keratinocytes using various retroviral vectors (Jacków et al., 2016, Georgiadis et al., 2016, Goto et al., 2006) were successfully used. Currently, clinical trials of such approaches are in different phases of study (NCT01263379, NCT02810951).

The KRT5 and KRT14 genes encode keratin 5 and keratin 14 proteins, respectively. Keratins are a group of rigid fibrous proteins that determine the structure of skin, hair and nails. Keratin 5 is produced in keratinocytes. Keratin 5 with keratin 14 form molecules called keratin intermediate filaments. These filaments are collected in a mesh structure necessary for the attachment of keratinocytes and connection between epidermis and underlying skin layers. A network of keratin intermediate filaments provides strength and elasticity to the skin and protects it from damage due to friction and other mechanical stresses. Mutations in KRT5 and KRT14 cause about 75% of cases of epidermolysis bullosa, and disease severity depends on the region of gene mutation (Bolling, Lemmink, Jansen, & Jonkman, 2011; Pfendner et al., 2016). In KRT5 gene knockout mice, there is a complete lack of connection between dermis and epidermis (Cao et al., 2001; Peters, Kirfel, Büssow, Vidal & Magin, 2001).

In addition to epidermolysis bullosa, mutations in KRT5 and KRT14 genes cause such diseases as reticulate pigmented anomaly of the flexures (Dowling-Degos disease), reticular pigmented dermatopathy (Oberst-Lehn-Hauss pigmented dermatopathy), Naegeli-Franceschetti-Jadassohn syndrome (Coulombe P A et al., 2017).

The LAMB3 gene encodes the laminin beta 3. i.e. subunit laminin. Laminins are a group of proteins that regulate cell growth, movement, and adhesion. They are also involved in the formation and organization of basement membranes that constitute thin, sheet-like structures that separate and support cells in many tissues. Laminin is a major component of fibres called anchoring filaments that connect the two layers of the basement membrane and help form a single skin structure, and mutations in the LAMB3 gene cause epidermolysis bullosa. Studies demonstrate that laminin also has several other functions. This protein appears to be important in wound healing and enamel genesis.

It is known from literature that mutations in the LAMB3 gene can cause hereditary brown enamel (Poulter et al., 2014), and some rare alleles of this gene are associated with lipomatosis dolorosa and risk of type 2 diabetes (Jiao H et al., 2016).

Experimental work on a volunteer patient with epidermolysis bullosa showed that transplantation of cells transfected with a retroviral vector expressing the LAMB3 gene resulted in a significant improvement in the sites of administration that persisted for at least a year (Mavilio et al., 2006).

Phase I/II clinical trials show that injection of autologous genetically modified epithelial cells expressing the LAMB3 gene seems to be a promising approach to the treatment of epidermolysis bullosa (De Rosa L et al., 2013).

Thus, the background of the Invention suggests that mutations in KRT5, KRT14, LAMB3, and COL7A1 genes or insufficient expression of proteins encoded by these genes are associated with the development of a spectrum of diseases, including, but not limited to, connective tissue diseases, wound healing, inherited and acquired pathological processes, such as epidermolysis bullosa, and other adverse conditions to the body. This is why KRT5, KRT14, LAMB3, and COL7A1 genes are grouped within this patent. Genetic constructs that provide expression of proteins encoded by KRT5, KRT14, LAMB3, and COL7A1 genes can be used to develop drugs for the prevention and treatment of different diseases and pathological conditions.

Moreover, these data suggest that insufficient expression of proteins encoded by KRT5, KRT14, LAMB3, and COL7A1 genes included in the group of genes is associated not only with pathological conditions, but also with a predisposition to their development. Also, these data indicate that insufficient expression of these proteins may not appear explicitly in the form of a pathology that can be unambiguously described within the framework of existing clinical practice standards (for example, using the ICD code), but at the same time cause conditions that are unfavourable for humans and animals and associated with deterioration in the quality of life.

Analysis of approaches to increase the expression of therapeutic genes implies the practicability of use of different gene therapy vectors.

Gene therapy vectors are divided into viral, cell, and DNA vectors (Guideline on the quality, non-clinical and clinical aspects of gene therapy medicinal Products EMA/COL7A1/80183/2014). Recently, gene therapy has paid increasingly more attention to the development of non-viral gene delivery systems with plasmid vectors topping the list. Plasmid vectors are free of limitations inherent in cell and viral vectors. In the target cell, they exist as an episome without being integrated into the genome, while producing them is quite cheap, and there is no immune response or side effects caused by the administration of plasmid vectors, which makes them a convenient tool for gene therapy and prevention of the genetic diseases (DNA vaccination) (Li L, Petrovsky N.//Expert Rev Vaccines. 2016; 15(3):313-29).

However, limitations of plasmid vectors use in gene therapy are: 1) presence of antibiotic resistance genes for the production of constructs in bacterial strains; 2) the presence of various regulatory elements represented by sequences of viral genomes; 3) length of therapeutic plasmid vector that determines the efficiency of vector delivery to the target cell.

It is known that the European Medicines Agency deems it necessary to refrain from adding antibiotic resistance marker genes to newly engineered plasmid vectors for gene therapy (Reflection paper on design modifications of gene therapy medicinal products during development/14 Dec. 2011 EMA/COL7A1/GTWP/44236/2009 Committee for advanced therapies). This recommendation is primarily related to the potential danger of the DNA vector penetration or horizontal antibiotic resistance gene transfer into the cells of bacteria found in the body as part of normal or opportunistic microflora. Furthermore, the presence of antibiotic resistance genes significantly increases the length of DNA vector, which reduces the efficiency of its penetration into eukaryotic cells.

It is important to note that antibiotic resistance genes also make a fundamental contribution to the method of production of DNA vectors. If antibiotic resistance genes are present, strains for the production of DNA vectors are usually cultured in medium containing a selective antibiotic, which poses risk of antibiotic traces in insufficiently purified DNA vector preparations. Thus, production of DNA vectors for gene therapy without antibiotic resistance genes is associated with the production of strains with such distinctive feature as the ability for stable amplification of therapeutic DNA vectors in the antibiotic-free medium.

In addition, the European Medicines Agency recommends avoiding the presence of regulatory elements in therapeutic plasmid vectors to increase the expression of therapeutic genes (promoters, enhancers, post-translational regulatory elements) that constitute nucleotide sequences of genomes of various viruses (Draft Guideline on the quality, non-clinical and clinical aspects of gene therapy medicinal products, http://www.ema.europa.eu/docs/en_GB/document_library/Scientific_guideline/2015/05/WC500187020.pdf). Although these sequences can increase the expression level of the therapeutic transgene, however, they pose risk of recombination with the genetic material of wild-type viruses and integration into the eukaryotic genome. Moreover, the relevance of overexpression of the particular gene for therapy remains an unresolved issue.

The size of the therapy vector is also essential. It is known that modern plasmid vectors often have unnecessary, non-functional sites that increase their length substantially (Mairhofer J, Grabherr R.//Mol Biotechnol. 2008. 39(2):97-104). For example, ampicillin resistance gene in pBR322 series vectors, as a rule, consists of at least 1000 bp, which is more than 20% of the length of the vector itself. A reverse relationship between the vector length and its ability to penetrate into eukaryotic cells is observed; DNA vectors with a small length effectively penetrate into human and animal cells. For example, in a series of experiments on transfection of HeLa cells with 383-4548 bp DNA vectors it was shown that the difference in penetration efficiency can be up to two orders of magnitude (100 times different) (Hornstein B D et al.//PLOS ONE. 2016; 11(12): e0167537.).

Thus, when selecting a DNA vector, for reasons of safety and maximum effectiveness, preference should be given to those constructs that do not contain antibiotic resistance genes, the sequences of viral origin and length of which allows for the effective penetration into eukaryotic cells. A strain for production of such DNA vector in quantities sufficient for the purposes of gene therapy should ensure the possibility of stable DNA vector amplification using antibiotic-free nutrient media.

Example of usage of the recombinant DNA vectors for gene therapy is the method of producing a recombinant vector for genetic immunisation (U.S. Pat. No. 9,550,998 B2. The plasmid vector is a supercoiled plasmid DNA vector that is used for the expression of cloned genes in human and animal cells. The vector contains an origin of replication, regulatory elements comprising human cytomegalovirus promoter and enhancer, and regulatory sequences from the human T-cell lymphotropic virus.

The vector is accumulated in a dedicated E. coli strain free of antibiotics through antisense complementation of sacB gene inserted into the strain by means of bacteriophage. The disadvantage of this invention is the presence of regulatory elements in the composition of DNA vector that constitute sequences of viral genomes.

The following applications are prototypes of this invention with regard to the use of gene therapy approaches to increase the expression level of genes from the group of KRT5, KRT14, LAMB3, and COL7A1 genes.

Application No. 20020064876A1 describes the invention based on the injection of complex oligonucleotide consisting of RNA and DNA for the correction of expression disorders of genes from the group of KRT5, KRT14, LAMB3, and COL7A1 genes, for the correction of pathological conditions of the skin, including hereditary diseases, such as epidermolysis bullosa. The disadvantage of this invention is a method for correcting disorders in genes that changes the nucleotide sequence of these genes, whereas not all disorders are limited to mutations in the coding regions of genes, and may also be associated with insufficient functional activity that is not due to mutations or such mutations that cannot be corrected by this method.

### DISCLOSURE OF THE INVENTION

The purpose of this invention is to construct the gene therapy DNA vectors in order to increase the expression level of a group of KRT5, KRT14, LAMB3, and COL7A1 genes in human and animal organisms that combine the following properties:
I)
Efficiency of gene therapy DNA vector in order to increase the expression level of therapeutic genes in eukaryotic cells.
II)
Possibility of safe use in gene therapy of human beings and animals due to the absence of regulatory elements representing the nucleotide sequences of viral genomes in the gene therapy DNA vector.
III)
Possibility of safe use in the gene therapy of human beings and animals due to the absence of antibiotic resistance genes in the gene therapy DNA vector.
IV)
Producibility and constructability of gene therapy DNA vector on an industrial scale.

Item II and III are provided for herein in line with the recommendations of the state regulators for gene therapy medicines and, specifically, the requirement of the European Medicines Agency to refrain from adding antibiotic resistance marker genes to newly engineered plasmid vectors for gene therapy (Reflection paper on design modifications of gene therapy medicinal products during development/14 Dec. 2011, EMA/COL7A1/GTWP/44236/2009 Committee for advanced therapies) and refrain from adding viral genomes to newly engineered plasmid vectors for gene therapy (Guideline on the quality, non-clinical and clinical aspects of gene therapy medicinal products/23 Mar. 2015, EMA/COL7A1/80183/2014, Committee for Advanced Therapies).

The purpose of the invention also includes the construction of strains carrying these gene therapy DNA vectors for the development and production of these gene therapy DNA vectors on an industrial scale.

The specified purpose is achieved by using the produced gene therapy DNA vector based on the gene therapy DNA vector VTvaf17 for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel, while the gene therapy DNA vector VTvaf17-KRT5 contains the coding region of KRT5 therapeutic gene cloned to gene therapy DNA vector VTvaf17 with the nucleotide sequence SEQ ID No. 1, the gene therapy DNA vector VTvaf17-KRT14 contains the coding region of KRT14 therapeutic gene cloned to gene therapy DNA vector VTvaf17 with the nucleotide sequence SEQ ID No. 2, the gene therapy DNA vector VTvaf17-LAMB3 contains the coding region of LAMB3 therapeutic gene cloned to gene therapy DNA vector VTvaf17 with the nucleotide sequence SEQ ID No. 3, the gene therapy DNA vector VTvaf17-COL7A1 contains the coding region of COL7A1 therapeutic gene cloned to gene therapy DNA vector VTvaf17 with the nucleotide sequence SEQ ID No. 4. Each of the constructed gene therapy DNA vectors, namely VTvaf17-KRT5, or VTvaf17-KRT14, or VTvaf17-LAMB3, or VTvaf17-COL7A1 due to the limited size of VTvaf17 vector part not exceeding 3200 bp has the ability to efficiently penetrate into human and animal cells and express the KRT5, or KRT14, or LAMB3, or COL7A1.

Each of the constructed gene therapy DNA vectors, namely VTvaf17-KRT5, or VTvaf17-KRT14, or VTvaf17-LAMB3, or VTvaf17-COL7A1 uses nucleotide sequences that are not antibiotic resistance genes, virus genes, or regulatory elements of viral genomes as the structure elements, which ensures its safe use for gene therapy in humans and animals.

A method of gene therapy DNA vector production based on gene therapy DNA vector VTvaf17 carrying the KRT5, KRT14, LAMB3, and COL7A1 therapeutic gene was also developed that involves obtaining each of gene therapy DNA vectors: VTvaf17-KRT5, or VTvaf17-KRT14, or VTvaf17-LAMB3, or VTvaf17-COL7A1 as follows: the coding region of the KRT5, or KRT14, or LAMB3, or COL7A1 therapeutic gene is cloned to gene therapy DNA vector VTvaf17, and gene therapy DNA vector VTvaf17-KRT5, SEQ ID No. 1, or VTvaf17-KRT14, SEQ ID No. 2 or VTvaf17-LAMB3, SEQ ID No. 3, or VTvaf17-COL7A1, SEQ ID No. 4, respectively, is obtained, while the coding region of the KRT5, or KRT14, or LAMB3, or COL7A1 therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvaf17 is performed by BamHII and HindIII, or SalI and EcoRI restriction sites, while the selection is performed without antibiotics,
at the same time, the following oligonucleotides produced for this purpose are used during gene therapy DNA vector VTvaf17-KRT5, SEQ ID No. 1 production for the reverse transcription reaction and PCR amplification:

KRT5_F
TTTGGATCCACCATGTCTCGCCAGTCAAGTGTGTCCTTC,
KRT5_R
AATAAGCTTCTAGCTCTTGAAGCTCTTCCGGGAGG,

and the cleaving of amplification product and cloning of the coding region of KRT5 gene to gene therapy DNA vector VTvaf17 is performed by BamHII and HindIII restriction endonucleases,
at the same time, the following oligonucleotides produced for this purpose are used during gene therapy DNA vector VTvaf17-KRT14, SEQ ID No. 2 production for the reverse transcription reaction and PCR amplification:

KRT14_F
TTTGGATCCACCATGACCACCTGCAGCCGCCAG,
KRT14_R
AATAAGCTTTCAGTTCTTGGTGCGAAGGACCTGC,

and the cleaving of amplification product and cloning of the coding region of KRT14 gene to gene therapy DNA vector VTvaf17 is performed by BamHII and HindIII restriction endonucleases,
at the same time, the following oligonucleotides produced for this purpose are used during gene therapy DNA vector VTvaf17-LAMB3, SEQ ID No. 3 production for the reverse transcription reaction and PCR amplification:

LAMB3_F
TTAGTCGACCACCATGAGACCATTCTTCCTCTTG,
LAMB3_R
ATAGAATTCACTTGCAGGTGGCATAGTAGAG,

and the cleaving of amplification product and cloning of the coding region of LAMB3 gene to gene therapy DNA vector VTvaf17 is performed by SalI and EcoRI restriction endonucleases,
at the same time, the following oligonucleotides produced for this purpose are used during gene therapy DNA vector VTvaf17-COL7A1, SEQ ID No. 4 production for the reverse transcription reaction and PCR amplification:

COL7A1_F
ATCGTCGACCACCATGACGCTGCGGCTTCTGGT,
COL7A1_R
ATAGAATTCAGTCCTGGGCAGTACCTGTC,

and the cleaving of amplification product and cloning of the coding region of COL7A1 gene to gene therapy DNA vector VTvaf17 is performed by SalI and EcoRI restriction endonucleases.

A method of use of the gene therapy DNA vector based on gene therapy DNA vector VTvaf17 carrying KRT5, KRT14, LAMB3, and COL7A1 therapeutic gene for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel was developed that involves transfection of the cells of patient or animal organs and tissues with the selected gene therapy DNA vector carrying the therapeutic gene based on gene therapy DNA vector VTvaf17, or several selected gene therapy DNA vectors carrying therapeutic genes based on gene therapy DNA vector VTvaf17, from the group of constructed gene therapy DNA vectors carrying therapeutic genes based on gene therapy DNA vector VTvaf17 and/or injection of autologous cells of said patient or animal transfected by the selected gene therapy DNA vector carrying therapeutic gene based on gene therapy DNA vector VTvaf17 or several selected gene therapy DNA vectors carrying the therapeutic genes based on gene therapy DNA vector VTvaf17 from the constructed gene therapy DNA vectors carrying therapeutic genes based on gene therapy DNA vector VTvaf17 into the organs and tissues of the same patient or animal and/or the injection of the selected gene therapy DNA vector carrying therapeutic gene based on gene therapy DNA vector VTvaf17 or several selected gene therapy DNA vectors carrying therapeutic genes based on gene therapy DNA vector VTvaf17 from the group of constructed gene therapy DNA vectors carrying therapeutic genes based on gene therapy DNA vector VTvaf17 into the organs and tissues of the same patient or animal, or the combination of the indicated methods.

A method of production of strain for construction of a gene therapy DNA vector for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel was developed that involves making electrocompetent cells of Escherichia coli strain SCS110-AF and subjecting these cells to electroporation with gene therapy DNA vector VTvaf17-KRT5, or gene therapy DNA vector VTvaf17-KRT14, or gene therapy DNA vector VTvaf17-LAMB3, or gene therapy DNA vector VTvaf17-COL7A1. After that, the cells are poured into agar plates (Petri dishes) with a selective medium containing yeastrel, peptone, 6% sucrose, and 10 μg/ml of chloramphenicol, and as a result, Escherichia coli strain SCS110-AF/VTvaf17-KRT5 or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 is obtained.

Escherichia coli strain SCS110-AF/VTvaf17-KRT5 carrying the gene therapy DNA vector VTvaf17-KRT5 for production thereof allowing for antibiotic-free selection during gene therapy DNA vector production, or Escherichia coli strain SCS110-AF/VTvaf17-KRT14 carrying the gene therapy DNA vector VTvaf17-KRT14 for production thereof allowing for antibiotic-free selection during gene therapy DNA vector production, or Escherichia coli strain SCS110-AF/VTvaf17-LAMB3 carrying the gene therapy DNA vector VTvaf17-LAMB3 for production thereof allowing for antibiotic-free selection during gene therapy DNA vector production, or Escherichia coli strain SCS110-AF/VTvaf17-COL7A1 carrying the gene therapy DNA vector VTvaf17-COL7A1 for production thereof allowing for antibiotic-free selection during gene therapy DNA vector production is claimed for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel.

A method of production on an industrial scale of gene therapy DNA vector based on gene therapy DNA vector VTvaf17 carrying the KRT5, or KRT14, or LAMB3, or COL7A1 therapeutic gene for treatment for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel was developed that involves production of gene therapy DNA vector VTvaf17-KRT5, or gene therapy DNA vector VTvaf17-KRT14, or gene therapy DNA vector VTvaf17-LAMB3, or gene therapy DNA vector VTvaf17-COL7A1 by inoculating a culture flask containing the prepared medium with seed culture selected from Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1, then the cell culture is incubated in an incubator shaker and transferred to an industrial fermenter, then grown to a stationary phase, then the fraction containing the target DNA product is extracted, multi-stage filtered, and purified by chromatographic methods.

The essence of the invention is explained in the drawings, where:

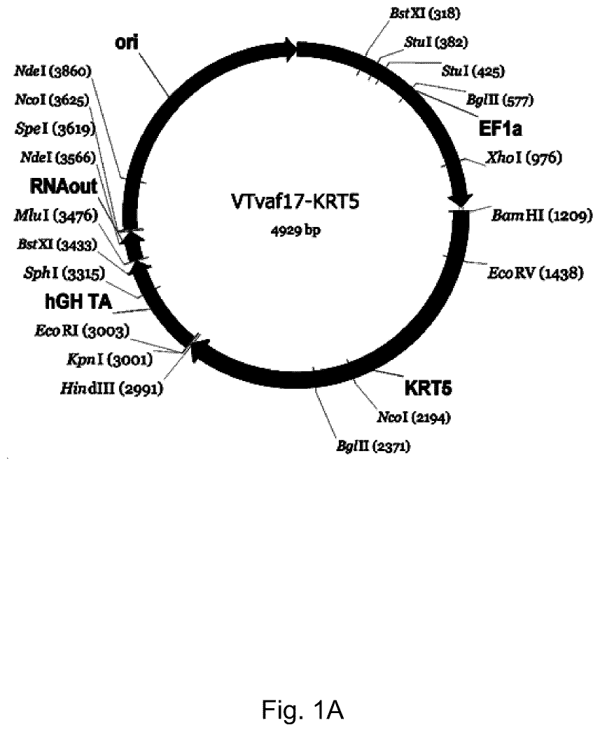

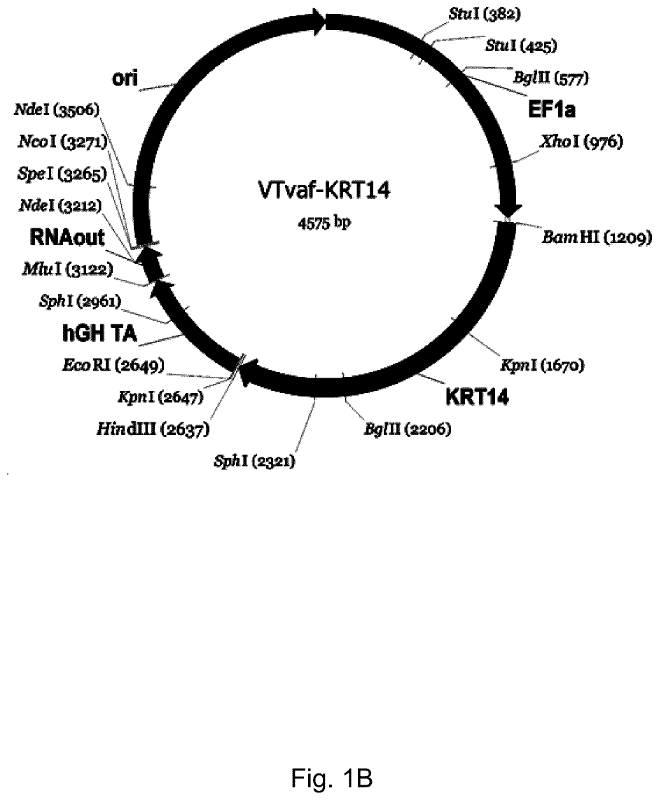

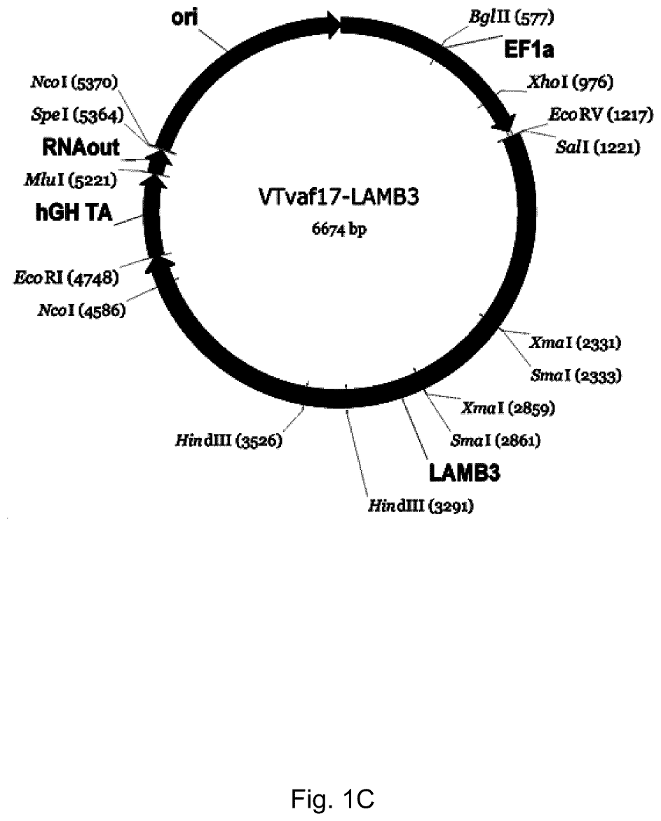

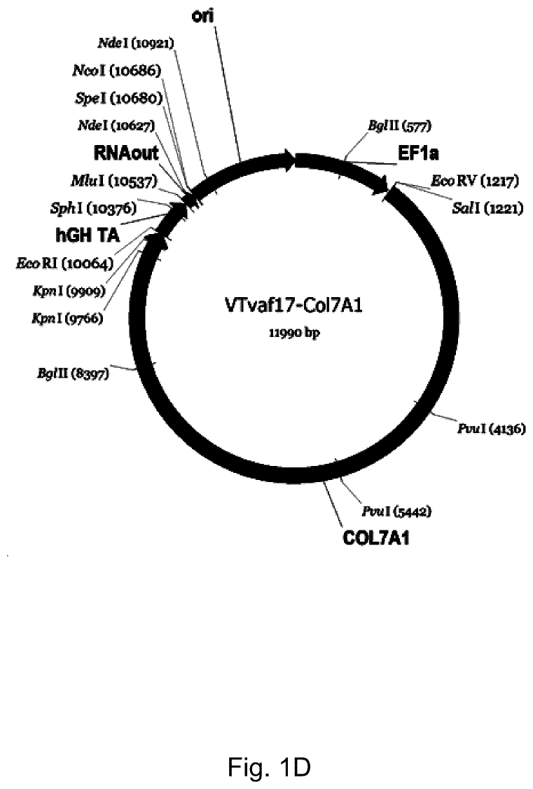

FIG. 1

shows the structure of gene therapy DNA vector VTvaf17 carrying the therapeutic gene selected from the group of KRT5, KRT14, LAMB3, and COL7A1 genes that constitutes a circular double-stranded DNA molecule capable of autonomous replication in Escherichia coli cells.

FIG. 1 shows the structures corresponding to:
A—gene therapy DNA vector VTvaf17-KRT5,
B—gene therapy DNA vector VTvaf17-KRT144,
C—gene therapy DNA vector VTvaf17-LAMB3,
D—gene therapy DNA vector VTvaf17-COL7A1.

The following structural elements of the vector are indicated in the structures:
EF1a—the promoter region of human elongation factor EF1A with an intrinsic enhancer contained in the first intron of the gene. It ensures efficient transcription of the recombinant gene in most human tissues,

The reading frame of the therapeutic gene corresponding to the coding region of the KRT5 (FIG. 1A), or KRT14 (FIG. 1B), or LAMB3 (FIG. 1C), or COL7A1 (FIG. 1D), respectively,
hGH TA—the transcription terminator and the polyadenylation site of the human growth factor gene,
ori—the origin of replication for autonomous replication with a single nucleotide substitution to increase plasmid production in the cells of most Escherichia coli strains,
RNA-out—the regulatory element RNA-out of transposon Tn 10 allowing for antibiotic-free positive selection in case of the use of Escherichia coli strain SCS 110-AF.

Unique restriction sites are marked.

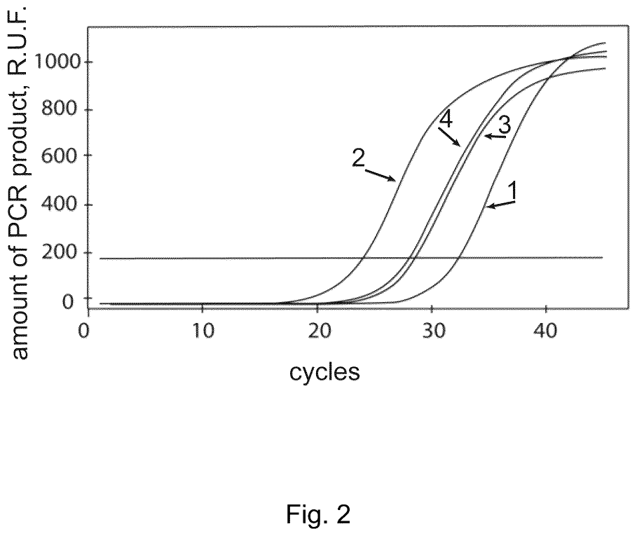

FIG. 2

shows diagrams of cDNA amplicon accumulation of the therapeutic gene, namely the KRT5 gene, in HDFa primary human dermal fibroblast cell culture (ATCC PCS-201-012) before its transfection and 48 hours after transfection of these cells with gene therapy DNA vector VTvaf17-KRT5 in order to assess the ability to penetrate into eukaryotic cells and functional activity, i.e. expression of the therapeutic gene at the mRNA level.

Curves of accumulation of amplicons during the reaction are shown in FIG. 2 corresponding to:
1—cDNA of KRT5 gene in HDFa primary human dermal fibroblast cell culture before transfection with DNA vector VTvaf17-KRT5,
2—cDNA of KRT5 gene in HDFa primary human dermal fibroblast cell culture after transfection with DNA vector VTvaf17-KRT5,
3—cDNA of B2M gene in HDFa primary human dermal fibroblast cell culture before transfection with DNA vector VTvaf17-KRT5,
4—cDNA of B2M gene in HDFa primary human dermal fibroblast cell culture after transfection with DNA vector VTvaf17-KRT5.

B2M (beta-2-microglobuline) gene listed in the GenBank database under number NM 004048.2 was used as a reference gene.

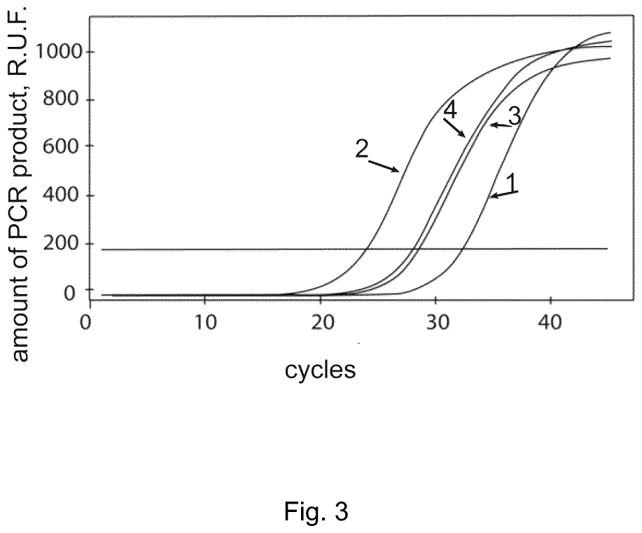

FIG. 3

shows diagrams of cDNA amplicon accumulation of the therapeutic gene, namely the KRT14 gene, in HEKa primary human epidermal keratinocyte cell culture (ATCC PCS-200-011) before its transfection and 48 hours after transfection of these cells with gene therapy DNA vector VTvaf17-KRT14 in order to assess the ability to penetrate into eukaryotic cells and functional activity, i.e. expression of the therapeutic gene at the mRNA level.

Curves of accumulation of amplicons during the reaction are shown in FIG. 3 corresponding to:
1—cDNA of KRT14 gene in HEKa primary human epidermal keratinocyte cell culture before transfection with DNA vector VTvaf17-KRT14,
2—cDNA of KRT14 gene in HEKa primary human epidermal keratinocyte cell culture after transfection with DNA vector VTvaf17-KRT14,
3—cDNA of B2M gene in HEKa primary human epidermal keratinocyte cell culture before transfection with DNA vector VTvaf17-KRT14,
4—cDNA of B2M gene in HEKa primary human epidermal keratinocyte cell culture after transfection with DNA vector VTvaf17-KRT14.

B2M (beta-2-microglobuline) gene listed in the GenBank database under number NM 004048.2 was used as a reference gene.

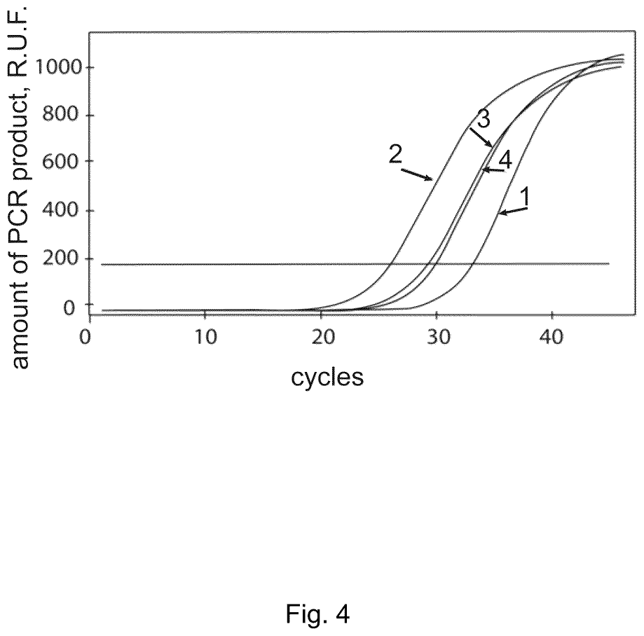

FIG. 4

shows diagrams of cDNA amplicon accumulation of the therapeutic gene, namely the LAMB3 gene, in Human Skeletal Myoblasts (HSKM) (GIBCO® Cat. A12555) before their transfection and 48 hours after transfection of these cells with the DNA vector VTvaf17-LAMB3 in order to assess the ability to penetrate into eukaryotic cells and functional activity, i.e. expression of the therapeutic gene at the mRNA level.

Curves of accumulation of amplicons during the reaction are shown in FIG. 4 corresponding to:
1—cDNA of LAMB3 gene in HSKM human skeletal myoblasts before transfection with DNA vector VTvaf17-LAMB3,
2—cDNA of LAMB3 gene in HSKM human skeletal myoblasts after transfection with DNA vector VTvaf17-LAMB3,
3—cDNA of B2M gene in HSKM human skeletal myoblasts before transfection with DNA vector VTvaf17-LAMB3,
4—cDNA of B2M gene in HSKM human skeletal myoblasts after transfection with DNA vector VTvaf17-LAMB3.

B2M (beta-2-microglobuline) gene listed in the GenBank database under number NM 004048.2 was used as a reference gene.

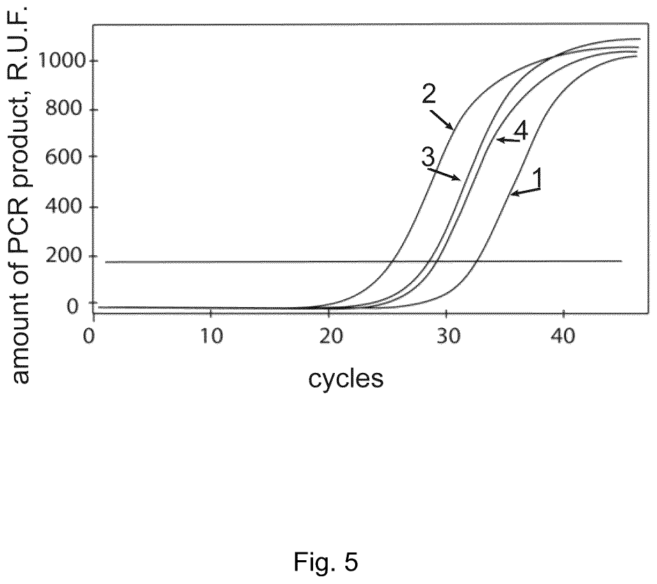

FIG. 5

shows diagrams of cDNA amplicon accumulation of the therapeutic gene, namely the COL7A1 gene, in Primary Umbilical Vein Endothelial Cells; Normal, Human (HUVEC) (ATCC® PCS-100-010™) before their transfection and 48 hours after transfection of these cells with the DNA vector VTvaf17-COL7A1 in order to assess the ability to penetrate into eukaryotic cells and functional activity, i.e. expression of the therapeutic gene at the mRNA level.

Curves of accumulation of amplicons during the reaction are shown in FIG. 5 corresponding to:
1—cDNA of COL7A1 gene in HUVEC human primary umbilical vein endothelial cell line before transfection with DNA vector VTvaf17-COL7A1,
2—cDNA of COL7A1 gene in HUVEC human primary umbilical vein endothelial cell line after transfection with DNA vector VTvaf17-COL7A1,
3—cDNA of B2M gene in HUVEC human primary umbilical vein endothelial cell line before transfection with DNA vector VTvaf17-COL7A1,
4—cDNA of B2M gene in HUVEC human primary umbilical vein endothelial cell line after transfection with DNA vector VTvaf17-COL7A1.

B2M (beta-2-microglobuline) gene listed in the GenBank database under number NM 004048.2 was used as a reference gene.

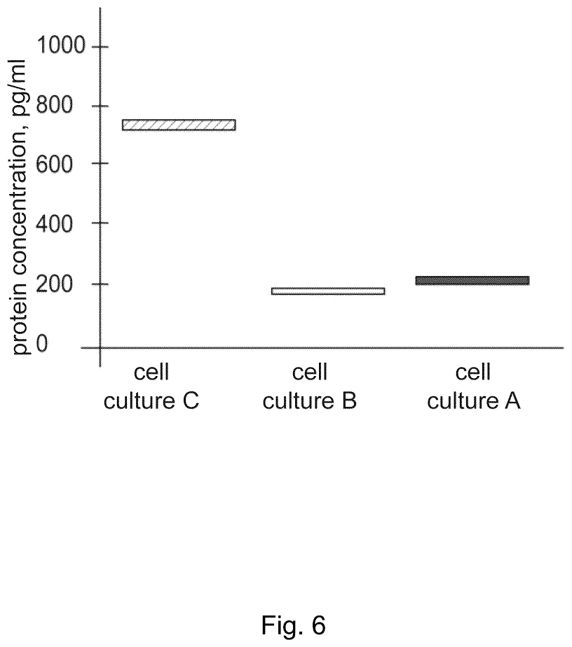

FIG. 6

shows the plot of KRT5 protein concentration in the cell lysate of HDFa human primary dermal fibroblasts (ATCC PCS-201-01) after transfection of these cells with DNA vector VTvaf17-KRT5 in order to assess the functional activity, i.e. expression at the protein level based on the KRT5 protein concentration change in the cell lysate.

The following elements are indicated in FIG. 6:
culture A—HDFa human primary dermal fibroblast cell culture transfected with aqueous dendrimer solution without plasmid DNA (reference),
culture B—HDFa primary human dermal fibroblast cell culture transfected with DNA vector VTvaf17,
culture C—HDFa human primary dermal fibroblast cell culture transfected with DNA vector VTvaf17-KRT5.

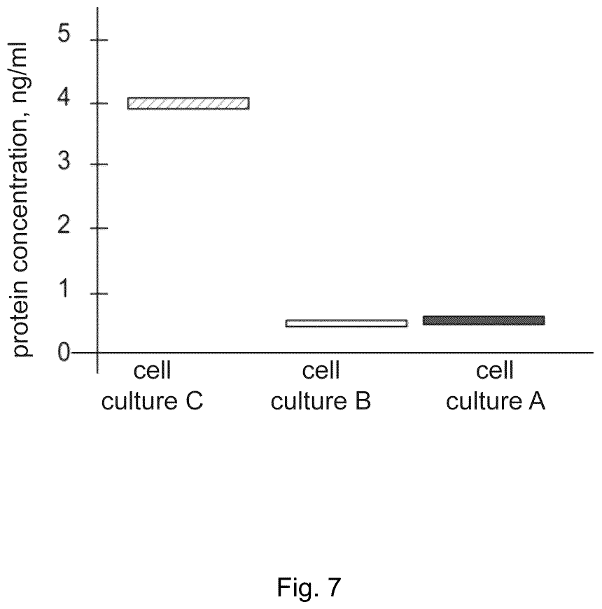

FIG. 7

shows the plot of KRT14 protein concentration in the lysate of HEKa primary human epidermal keratinocyte cells (ATCC PCS-200-01) after transfection of these cells with gene therapy DNA vector VTvaf17-KRT14 in order to assess the functional activity, i.e. the therapeutic gene expression at the protein level, and the possibility of increasing the level of protein expression by gene therapy DNA vector based on gene therapy vector VTvaf17 carrying the KRT14 therapeutic gene.

The following elements are indicated in FIG. 7:
culture A—HEKa primary human epidermal keratinocyte cell culture transfected with aqueous dendrimer solution without plasmid DNA (reference),
culture B—HEKa primary human epidermal keratinocyte cell culture transfected with DNA vector VTvaf17,
culture C—HEKa primary human epidermal keratinocyte cell culture transfected with DNA vector VTvaf17-KRT14.

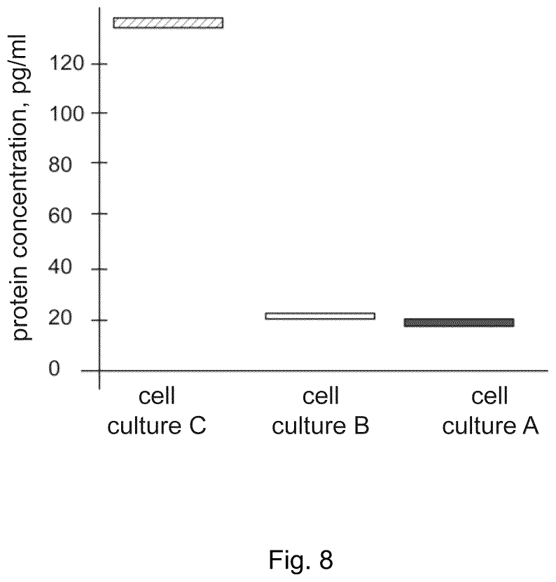

FIG. 8

shows the plot of LAMB3 protein concentration in the lysate of Human Skeletal Myoblasts (HSKM) (GIBCO® Cat. A12555) after transfection of these cells with gene therapy DNA vector VTvaf17-LAMB3 in order to assess the functional activity, i.e. the therapeutic gene expression at the protein level, and the possibility of increasing the level of protein expression by gene therapy DNA vector based on gene therapy vector VTvaf17 carrying the LAMB3 therapeutic gene.

The following elements are indicated in FIG. 8:
culture A—HSKM human primary skeletal muscle myoblast cells transfected with aqueous dendrimer solution without plasmid DNA (reference),
culture B—HSKM human primary skeletal muscle myoblast cells transfected with DNA vector VTvaf17,
culture C—HSKM human primary skeletal muscle myoblast cells transfected with DNA vector VTvaf17-LAMB3.

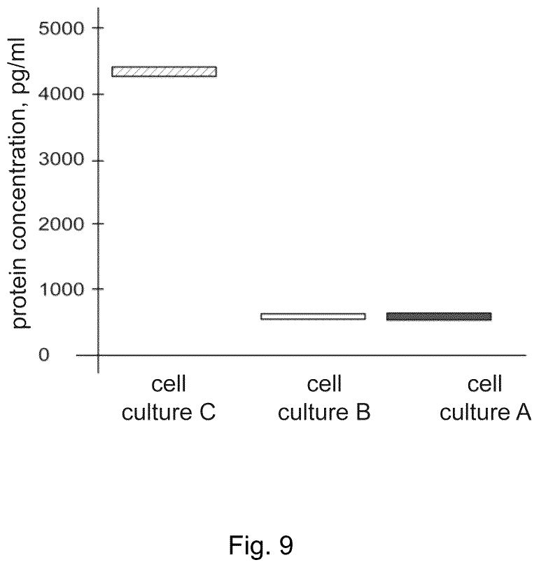

FIG. 9

shows the plot of COL7A1 protein concentration in the cell lysate of Primary Umbilical Vein Endothelial Cells; Normal, Human (HUVEC) (ATCC® PCS-100-010™) after transfection of these cells with gene therapy DNA vector VTvaf17-COL7A1 in order to assess the functional activity, i.e. the therapeutic gene expression at the protein level, and the possibility of increasing the level of protein expression by gene therapy DNA vector based on gene therapy DNA vector VTvaf17 carrying the COL7A1 therapeutic gene.

The following elements are indicated in FIG. 9:
culture A—HUVEC human umbilical vein endothelial cell culture transfected with aqueous dendrimer solution without plasmid DNA (reference),
culture B—HUVEC human umbilical vein endothelial cell culture transfected with DNA vector VTvaf17,
culture C—HUVEC human umbilical vein endothelial cell culture transfected with DNA vector VTvaf17-COL7A1.

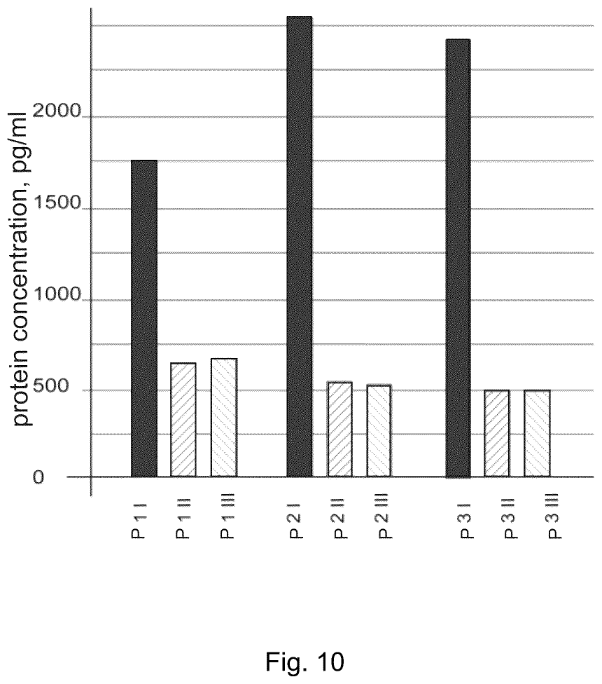

FIG. 10

shows the plot of COL7A1 protein concentration in the skin biopsy specimens of three patients after injection of gene therapy DNA vector VTvaf17-COL7A1 into the skin of these patients in order to assess the functional activity, i.e. the expression of the therapeutic gene at the protein level, and the possibility of increasing the level of protein expression using gene therapy DNA vector based on gene therapy vector VTvaf17 carrying the COL7A1 therapeutic gene.

The following elements are indicated in FIG. 10:
P1I—patient P1 skin biopsy in the region of injection of gene therapy DNA vector VTvaf17-COL7A1,
P1II—patient P1 skin biopsy in the region of injection of gene therapy DNA vector VTvaf17 (placebo),
P1III—patient P1 skin biopsy from intact site,
P2I—patient P2 skin biopsy in the region of injection of gene therapy DNA vector VTvaf17-COL7A1,
P2II—patient P2 skin biopsy in the region of injection of gene therapy DNA vector VTvaf17 (placebo),
P2III—patient P2 skin biopsy from intact site,
P3I—patient P3 skin biopsy in the region of injection of gene therapy DNA vector VTvaf17-COL7A1,
P3II—patient P3 skin biopsy in the region of injection of gene therapy DNA vector VTvaf17 (placebo),
P3III—patient P3 skin biopsy from intact site.

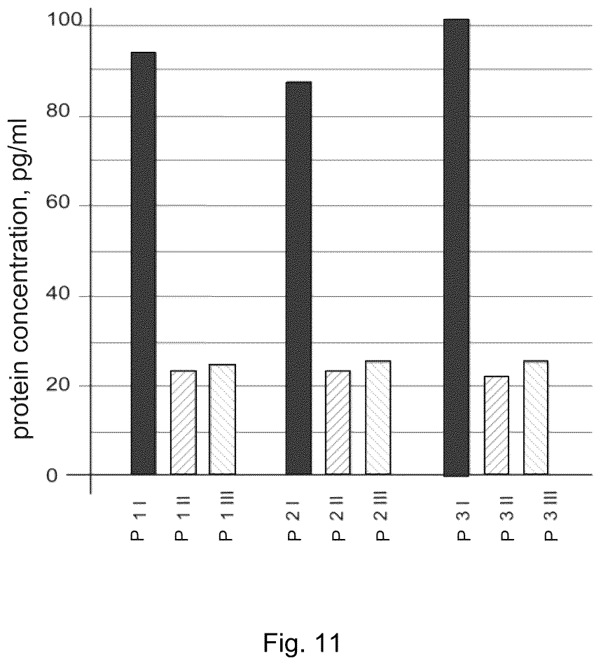

FIG. 11

shows the plot of LAMB3 protein concentration in the gastrocnemius muscle biopsy specimens of three patients after injection of gene therapy DNA vector VTvaf17-LAMB3 into the gastrocnemius muscle of these patients in order to assess the functional activity, i.e. the therapeutic gene expression at the protein level, and the possibility of increasing the level of protein expression using gene therapy DNA vector based on gene therapy vector VTvaf17 carrying the LAMB3 therapeutic gene.

The following elements are indicated in FIG. 11:
P1I—patient P1 gastrocnemius muscle biopsy in the region of injection of gene therapy DNA vector VTvaf17-LAMB3,
P1II—patient P1 gastrocnemius muscle biopsy in the region of injection of gene therapy DNA vector VTvaf17 (placebo),
P1III—patient P1 gastrocnemius muscle biopsy from intact site,
P2I—patient P2 gastrocnemius muscle biopsy in the region of injection of gene therapy DNA vector VTvaf17-LAMB3,
P2II—patient P2 gastrocnemius muscle biopsy in the region of injection of gene therapy DNA vector VTvaf17 (placebo),
P2III—patient P2 gastrocnemius muscle biopsy from intact site,
P3I—patient P3 gastrocnemius muscle biopsy in the region of injection of gene therapy DNA vector VTvaf17-LAMB3,
P3II—patient P3 gastrocnemius muscle biopsy in the region of injection of gene therapy DNA vector VTvaf17 (placebo),
P3III—patient P3 gastrocnemius muscle biopsy from intact site.

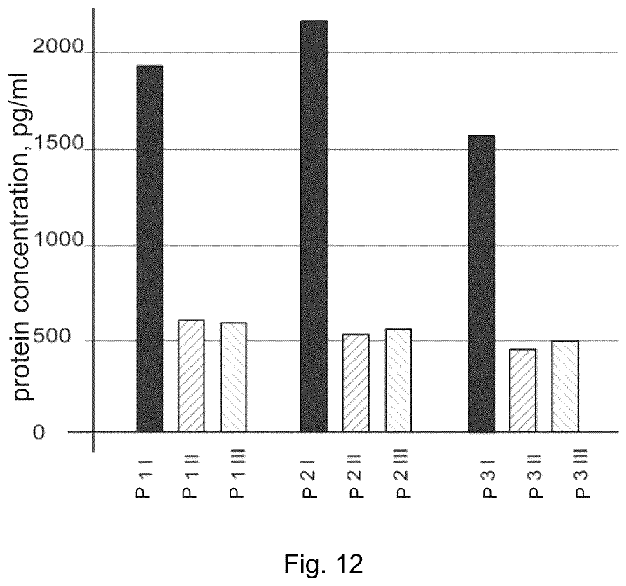

FIG. 12

shows the plot of KRT14 protein concentration in the skin biopsy samples of three patients after injection of gene therapy DNA vector VTvaf17-KRT14 into the skin of these patients in order to assess the functional activity, i.e. the expression of the therapeutic gene at the protein level, and the possibility of increasing the level of protein expression using gene therapy DNA vector based on gene therapy DNA vector VTvaf17 carrying the-KRT14 therapeutic gene.

The following elements are indicated in FIG. 12:
P1I—patient P1 skin biopsy in the region of injection of gene therapy DNA vector VTvaf17-KRT14,
P1II—patient P1 skin biopsy in the region of injection of gene therapy DNA vector VTvaf17 (placebo),
P1III—patient P1 skin biopsy from intact site,
P2I—patient P2 skin biopsy in the region of injection of gene therapy DNA vector VTvaf17-KRT14,
P2II—patient P2 skin biopsy in the region of injection of gene therapy DNA vector VTvaf17 (placebo),
P2III—patient P2 skin biopsy from intact site,
P3I—patient P3 skin biopsy in the region of injection of gene therapy DNA vector VTvaf17-KRT14,
P3II—patient P3 skin biopsy in the region of injection of gene therapy DNA vector VTvaf17 (placebo),
P3III—patient P3 skin biopsy from intact site.

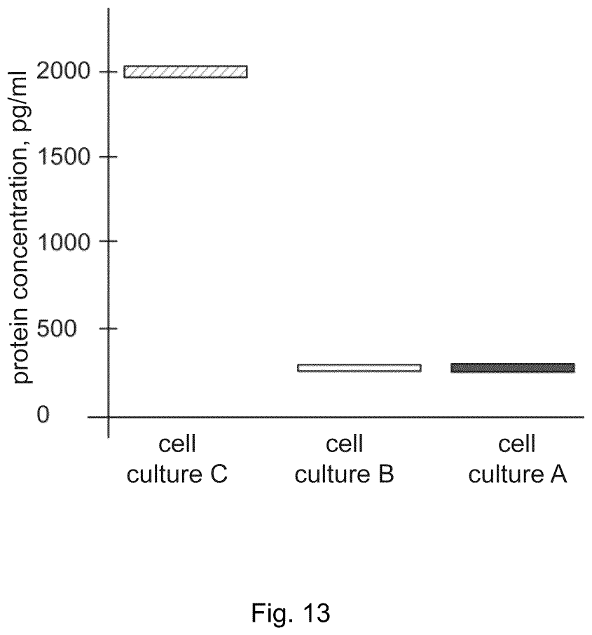

FIG. 13

shows the plot of KRT14 protein concentration in human skin biopsy samples after subcutaneous injection of autologous fibroblast cell culture transfected with the gene therapy DNA vector VTvaf17-KRT14 in order to demonstrate the method of use by injecting autologous cells transfected with the gene therapy DNA vector VTvaf17-KRT14.

The following elements are indicated in FIG. 13:
P1C—patient P1 skin biopsy in the region of injection of autologous fibroblast culture of the patient transfected with gene therapy DNA vector VTvaf17-KRT14,
P1B—patient P1 skin biopsy in the region of injection of autologous fibroblasts of the patient transfected with gene therapy DNA vector VTvaf17,
P1A—patient P1 skin biopsy from intact site.

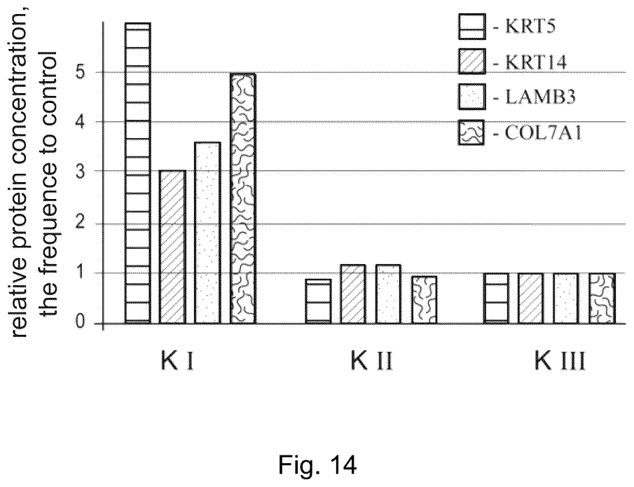

FIG. 14

shows the plot of concentration of the following proteins: human KRT5 protein, human KRT14 protein, human LAMB3 protein, and human COL7A1 protein in skin biopsy samples of three rats after injection of a mixture of the following gene therapy vectors: gene therapy DNA vector VTvaf17-KRT5, gene therapy DNA vector VTvaf17-KRT14, gene therapy DNA vector VTvaf17-LAMB3, and gene therapy DNA vector VTvaf17-COL7A1 in order to demonstrate the method of use of a mixture gene therapy DNA vectors.

The following elements are indicated in FIG. 14:
K1I—rat K1 skin biopsy sample in the region of injection of a mixture of gene therapy DNA vectors: VTvaf17-KRT5, VTvaf17-KRT14, VTvaf17-LAMB3, and VTvaf17-COL7A1,
K1II—rat K1 skin biopsy sample in the region of injection of gene therapy DNA vector VTvaf17 (placebo),
K1III—rat K1 skin biopsy sample of the reference intact site,
K2I—rat K2 skin biopsy sample in the region of injection of a mixture of gene therapy DNA vectors: VTvaf17-KRT5, VTvaf17-KRT14, VTvaf17-LAMB3, and VTvaf17-COL7A1,
K2II—rat K2 skin biopsy sample in the region of injection of gene therapy DNA vector VTvaf17 (placebo),
K2III—rat K2 skin biopsy sample of the reference intact site,
K31—rat K3 skin biopsy sample in the region of injection of a mixture of gene therapy DNA vectors: VTvaf17-KRT5, VTvaf17-KRT14, VTvaf17-LAMB3, and VTvaf17-COL7A1,
K3II—rat K3 skin biopsy sample in the region of injection of gene therapy DNA vector VTvaf17 (placebo),
K3III—rat K3 skin biopsy sample of the reference intact site.

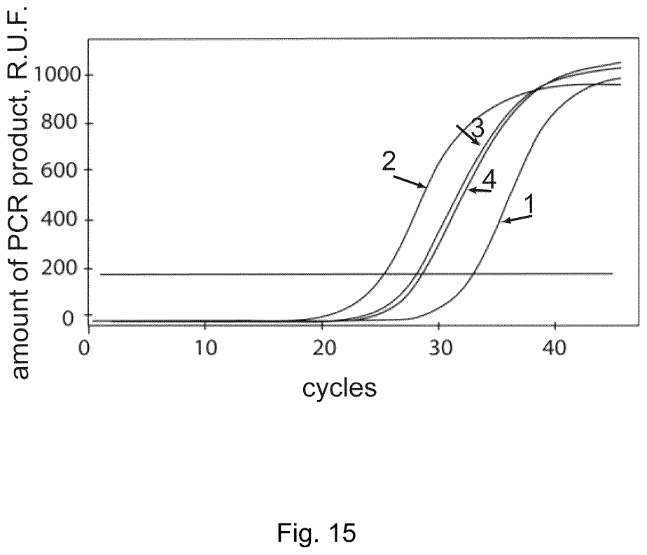

FIG. 15

shows diagrams of cDNA amplicon accumulation of the LAMB3 therapeutic gene in MDBK bovine kidney epithelial cells (NBL-1) (ATCC® CCL-22™) before and 48 hours after transfection of these cells with the DNA vector VTvaf17-LAMB3 in order to demonstrate the method of use by injecting the gene therapy DNA vector in animals.

Curves of accumulation of amplicons during the reaction are shown in FIG. 15 corresponding to:
1—cDNA of LAMB3 gene in MDBK cells before transfection with gene therapy DNA vector VTvaf17-LAMB3,
2—cDNA of LAMB3 gene in MDBK cells after transfection with gene therapy DNA vector VTvaf17-LAMB3,
3—cDNA of ACT gene in MDBK cells before transfection with gene therapy DNA vector VTvaf17-LAMB3,
4—cDNA of ACT gene in MDBK cells after transfection with gene therapy DNA vector VTvaf17-LAMB3.

Bull/cow actin gene (ACT) listed in the GenBank database under number AH001130.2 was used as a reference gene.

### EMBODIMENT OF THE INVENTION

Gene therapy DNA vectors carrying the human therapeutic genes designed to increase the expression level of these therapeutic genes in human and animal tissues were constructed based on 3165 bp DNA vector VTvaf17. The method of production of each gene therapy DNA vector carrying the therapeutic genes is to clone the protein coding sequence of the therapeutic gene selected from the group of the following genes: human KRT5 gene (encodes KRT5 protein), human KRT14 gene (encodes KRT14 protein), human LAMB3 gene (encodes LAMB3 protein), and human COL7A1 gene (encodes COL7A1 protein) to the polylinker of gene therapy DNA vector VTvaf17. It is known that the ability of DNA vectors to penetrate into eukaryotic cells is due mainly to the vector size. DNA vectors with the smallest size have higher penetration capability. Thus, the absence of elements in the vector that bear no functional load, but at the same time increase the vector DNA size is preferred. These features of DNA vectors were taken into account during the production of gene therapy DNA vectors based on gene therapy DNA vector VTvaf17 carrying the therapeutic gene selected from the group of KRT5, KRT14, LAMB3, and COL7A1 genes with no large non-functional sequences and antibiotic resistance genes in the vector, which, in addition to technological advantages and safe use, allowed for the significant reduction of size of the produced gene therapy DNA vector VTvaf17 carrying the therapeutic gene selected from the group of KRT5, KRT14, LAMB3, and COL7A1 genes. Thus, the ability of the obtained gene therapy DNA vector to penetrate into eukaryotic cells is due to its small length.

Each of the following gene therapy DNA vectors: VTvaf17-KRT5, or VTvaf17-KRT14, or VTvaf17-LAMB3, or VTvaf17-COL7A1 was produced as follows: the coding region of the therapeutic gene from the group of KRT5, KRT14, LAMB3, and COL7A1 genes was cloned to DNA vector VTvaf17, and gene therapy DNA vector VTvaf17-KRT5, SEQ ID No. 1, or VTvaf17-KRT14, SEQ ID No. 2, or VTvaf17-LAMB3, SEQ ID No. 3, or VTvaf17-COL7A1, SEQ ID No. 4, respectively, was obtained. The coding region of KRT5 gene (1776 bp), or KRT14 gene (1422 bp), or LAMB3 gene (3521 bp), or COL7A1 gene (8838 bp) was produced by extracting total RNA from the biological normal human tissue sample. The reverse transcription reaction was used for the synthesis of the first chain cDNA of human KRT5, KRT14, LAMB3, and COL7A1 genes. Amplification was performed using oligonucleotides produced for this purpose by the chemical synthesis method. The amplification product was cleaved by specific restriction endonucleases taking into account the optimal procedure for further cloning, and cloning to the gene therapy DNA vector VTvaf17 was performed by BamHI, SalI, EcoRI, and HindIII restriction sites located in the VTvaf17 vector polylinker. The selection of restriction sites was carried out in such a way that the cloned fragment entered the reading frame of expression cassette of the vector VTvaf17, while the protein coding sequence did not contain restriction sites for the selected endonucleases. Experts in this field realise that the methodological implementation of gene therapy DNA vector VTvaf17-KRT5, or VTvaf17-KRT14, or VTvaf17-LAMB3, or VTvaf17-COL7A1 production can vary within the framework of the selection of known methods of molecular gene cloning and these methods are included in the scope of this invention. For example, different oligonucleotide sequences can be used to amplify KRT5, or KRT14, or LAMB3, or COL7A1 gene, different restriction endonucleases or laboratory techniques, such as ligation independent cloning of genes.

Gene therapy DNA vector VTvaf17-KRT5, or VTvaf17-KRT14, or VTvaf17-LAMB3, or VTvaf17-COL7A1 has the nucleotide sequence SEQ ID No. 1, or SEQ ID No. 2, or SEQ ID No. 3, or SEQ ID No. 4, respectively. At the same time, degeneracy of genetic code is known to the experts in this field and means that the scope of this invention also includes variants of nucleotide sequences differing by insertion, deletion, or replacement of nucleotides that do not result in a change in the polypeptide sequence encoded by the therapeutic gene, and/or do not result in a loss of functional activity of the regulatory elements of VTvaf17 vector. At the same time, genetic polymorphism is known to the experts in this field and means that the scope of this invention also includes variants of nucleotide sequences of genes from the group of KRT5, KRT14, LAMB3, and COL7A1 genes that also encode different variants of the amino acid sequences of KRT5, KRT14, LAMB3, and COL7A1 proteins that do not differ from those listed in their functional activity under physiological conditions.

The ability to penetrate into eukaryotic cells and express functional activity, i.e. the ability to express the therapeutic gene of the obtained gene therapy DNA vector VTvaf17-KRT5, or VTvaf17-KRT14, or VTvaf17-LAMB3, or VTvaf17-COL7A1 is confirmed by introducing the obtained vector into eukaryotic cells and subsequent analysis of the expression of specific mRNA and/or protein product of the therapeutic gene. The presence of specific mRNA in cells into which the gene therapy DNA vector VTvaf17-KRT5, or VTvaf17-KRT14, or VTvaf17-LAMB3, or VTvaf17-COL7A1 was introduced shows the ability of the obtained vector to both penetrate into eukaryotic cells and express mRNA of the therapeutic gene. Furthermore, it is known to the experts in this field that the presence of mRNA gene is a mandatory condition, but not an evidence of the translation of protein encoded by the therapeutic gene. Therefore, in order to confirm properties of the gene therapy DNA vector VTvaf17-KRT5, or VTvaf17-KRT14, or VTvaf17-LAMB3, or VTvaf17-COL7A1 to express the therapeutic gene at the protein level in eukaryotic cells into which the gene therapy DNA vector was introduced, analysis of the concentration of proteins encoded by the therapeutic genes was carried out using immunological methods. The presence of KRT5, or KRT14, or LAMB3, or COL7A1 protein confirms the efficiency of expression of therapeutic genes in eukaryotic cells and the possibility of increasing the protein concentration using the gene therapy DNA vector based on gene therapy DNA vector VTvaf17 carrying the therapeutic gene selected from the group of KRT5, KRT14, LAMB3, and COL7A1 genes. Thus, in order to confirm the expression efficiency of the constructed gene therapy DNA vector VTvaf17-KRT5 carrying the therapeutic gene, namely the KRT5 gene, gene therapy DNA vector VTvaf17-KRT14 carrying the therapeutic gene, namely the KRT14 gene, gene therapy DNA vector VTvaf17-LAMB3 carrying the therapeutic gene, namely the LAMB3 gene, gene therapy DNA vector VTvaf17-COL7A1 carrying the therapeutic gene, namely the COL7A1 gene, the following methods were used:
A) real-time PCR, i.e. change in mRNA accumulation of therapeutic genes in human and animal cell lysate after transfection of different human and animal cell lines with gene therapy DNA vectors,
B) Enzyme-linked immunosorbent assay, i.e. change in the quantitative level of therapeutic proteins in the human cell lysate or medium after transfection of different human cell lines with gene therapy DNA vectors,
C) Enzyme-linked immunosorbent assay, i.e. change in the quantitative level of therapeutic proteins in the supernatant of human and animals tissue biopsy specimens after the injection of gene therapy DNA vectors into these tissues,
D) Enzyme-linked immunosorbent assay, i.e. change in the quantitative level of therapeutic proteins in the supernatant of human tissue biopsies after the injection of these tissues with autologous cells of this human transfected with gene therapy DNA vectors.

In order to confirm the practicability of use of the constructed gene therapy DNA vector VTvaf17-KRT5 carrying the therapeutic gene, namely the KRT5 gene, gene therapy DNA vector VTvaf17-KRT14 carrying the therapeutic gene, namely the KRT14 gene, gene therapy DNA vector VTvaf17-LAMB3 carrying the therapeutic gene, namely the LAMB3 gene, gene therapy DNA vector VTvaf17-COL7A1 carrying the therapeutic gene, namely the COL7A1 gene, the following was performed:
A) transfection of different human and animal cell lines with gene therapy DNA vectors,
B) injection of gene therapy DNA vectors into different human and animal tissues,
C) injection of a mixture of gene therapy DNA vectors into animal tissues,
D) injection of autologous cells transfected with gene therapy DNA vectors into human tissues.

These methods of use lack potential risks for gene therapy of humans and animals due to the absence of regulatory elements in the gene therapy DNA vector that constitute the nucleotide sequences of viral genomes and absence of antibiotic resistance genes in the gene therapy DNA vector as confirmed by the lack of regions homologous to the viral genomes and antibiotic resistance genes in the nucleotide sequences of gene therapy DNA vector VTvaf17-KRT5, or gene therapy DNA vector VTvaf17-KRT14, or gene therapy DNA vector VTvaf17-LAMB3, or gene therapy DNA vector VTvaf17-COL7A1 (SEQ ID No. 1, or SEQ ID No. 2, or SEQ ID No. 3, or SEQ ID No. 4, respectively).

It is known to the experts in this field that antibiotic resistance genes in the gene therapy DNA vectors are used to obtain these vectors in preparative quantities by increasing bacterial biomass in a nutrient medium containing a selective antibiotic. Within the framework of this invention, in order to ensure the safe use of gene therapy DNA vector VTvaf17 carrying KRT5, or KRT14, or LAMB3, or COL7A1 therapeutic genes, the use of selective nutrient media containing an antibiotic is not possible. A method for obtaining strains for production of these gene therapy vectors based on Escherichia coli strain SCS110-AF is proposed as a technological solution for obtaining the gene therapy DNA vector VTvaf17 carrying a therapeutic gene selected from the group of KRT5, KRT14, LAMB3, and COL7A1 genes in order to scale up the production of gene therapy vectors to an industrial scale. The method of Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 production involves production of competent cells of Escherichia coli strain SCS110-AF with the injection of gene therapy DNA vector VTvaf17-KRT5, or DNA vector VTvaf17-KRT14, or DNA vector VTvaf17-LAMB3, or DNA vector VTvaf17-COL7A1 into these cells, respectively, using transformation (electroporation) methods widely known to the experts in this field. The obtained Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 is used to produce the gene therapy DNA vector VTvaf17-KRT5, or VTvaf17-KRT14, or VTvaf17-LAMB3, or VTvaf17-COL7A1, respectively, allowing for the use of antibiotic-free media.

In order to confirm the production of Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1, transformation, selection, and subsequent biomass growth with extraction of plasmid DNA were performed.

To confirm the producibility and constructability and scale up of the production of gene therapy DNA vector VTvaf17-KRT5 carrying the therapeutic gene, namely KRT5 gene, gene therapy DNA vector VTvaf17-KRT14 carrying the therapeutic gene, namely KRT14 gene, gene therapy DNA vector VTvaf17-LAMB3 carrying the therapeutic gene, namely LAMB3 gene, gene therapy DNA vector VTvaf17-COL7A1 carrying the therapeutic gene, namely COL7A1 gene, to an industrial scale, the fermentation on an industrial scale of Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 each containing gene therapy DNA vector VTvaf17 carrying the therapeutic gene, namely KRT5, or KRT14, or LAMB3, or COL7A1 gene was performed.

The method of scaling the production of bacterial mass to an industrial scale for the isolation of gene therapy DNA vector VTvaf17 carrying the therapeutic gene selected from the group of KRT5, KRT14, LAMB3, and COL7A1 genes involves incubation of the seed culture of Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 in the antibiotic-free nutrient medium that provides suitable biomass accumulation dynamics. Upon reaching a sufficient amount of biomass in the logarithmic phase, the bacterial culture is transferred to an industrial fermenter and then grown to a stationary phase, then the fraction containing the therapeutic DNA product, i.e. gene therapy DNA vector VTvaf17-KRT5, or gene therapy DNA vector VTvaf17-KRT14, or gene therapy DNA vector VTvaf17-LAMB3, or gene therapy DNA vector VTvaf17-COL7A1, is extracted, multi-stage filtered, and purified by chromatographic methods. It is known to the experts in this field that culture conditions of strains, composition of nutrient media (except for antibiotic-free), equipment used, and DNA purification methods may vary within the framework of standard operating procedures depending on the particular production line, but known approaches to scaling, industrial production, and purification of DNA vectors using Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 fall within the scope of this invention.

The described disclosure of the invention is illustrated by examples of the embodiment of this invention.

The essence of the invention is explained in the following examples.

### Example 1

Production of gene therapy DNA vector VTvaf17-KRT5 carrying the therapeutic gene, namely the KRT5 gene.

Gene therapy DNA vector VTvaf17-KRT5 was constructed by cloning the coding region of KRT5 gene (1776 bp) to a 3165 bp DNA vector VTvaf17 by BamHI and HindIII restriction sites. The coding region of KRT5 gene (1776 bp) was obtained by isolating total RNA from the biological human tissue sample followed by reverse transcription reaction using commercial kit Mint-2 (Evrogen, Russia) and PCR amplification using the following oligonucleotides:
KRT5_F TTTGGATCCACCATGTCTCGCCAGTCAAGTGTGTCCTTC,
KRT5_R AATAAGCTTCTAGCTCTTGAAGCTCTTCCGGGAGG
and commercially available kit Phusion® High-Fidelity DNA Polymerase (New England Biolabs, USA).

Gene therapy DNA vector VTvaf17 was constructed by consolidating six fragments of DNA derived from different sources:
(a) the origin of replication was produced by PCR amplification of a region of commercially available plasmid pBR322 with a point mutation,
(b) EF1a promoter region was produced by PCR amplification of a site of human genomic DNA,
(c) hGH-TA transcription terminator was produced by PCR amplification of a site of human genomic DNA,
(d) the RNA-OUT regulatory site of transposon Tn10 was synthesised from oligonucleotides,
(e) kanamycin resistance gene was produced by PCR amplification of a site of commercially available human plasmid pET-28,
(f) the polylinker was produced by annealing two synthetic oligonucleotides.

PCR amplification was performed using the commercially available kit Phusion® High-Fidelity DNA Polymerase (New England Biolabs, USA) as per the manufacturer's instructions. The fragments have overlapping regions allowing for their consolidation with subsequent PCR amplification. Fragments (a) and (b) were consolidated using oligonucleotides Ori-F and EF1-R, and fragments (c), (d), and (e) were consolidated using oligonucleotides hGH-F and Kan-R. Afterwards, the produced fragments were consolidated by restriction with subsequent ligation by sites BamHI and NcoI. This resulted in a plasmid still devoid of the polylinker. To add it, the plasmid was cleaved by BamHI and EcoRI sites followed by ligation with fragment (f). Therefore, a 4182 bp vector was constructed carrying the kanamycin resistance gene flanked by SpeI restriction sites. Then this gene was cleaved by SpeI restriction sites and the remaining fragment was ligated to itself. This resulted in a 3165 bp gene therapy DNA vector VTvaf17 that is recombinant and allows for antibiotic-free selection.

The amplification product of the coding region of KRT5 gene and DNA vector VTvaf17 was cleaved by BamHI and HindIII restriction endonucleases (New England Biolabs, USA).

This resulted in a 4929 bp DNA vector VTvaf17-KRT5 with the nucleotide sequence SEQ ID No. 1 and general structure shown in FIG. 1A.

### Example 2

Production of gene therapy DNA vector VTvaf17-KRT14 carrying the therapeutic gene, namely the KRT14 gene.

Gene therapy DNA vector VTvaf17-KRT14 was constructed by cloning the coding region of KRT14 gene (1422 bp) to a 3165 bp DNA vector VTvaf17 by BamHI and HindIII restriction sites. The coding region of KRT14 gene (1422 bp) was obtained by isolating total RNA from the biological human tissue sample followed by reverse transcription reaction using commercial kit Mint-2 (Evrogen, Russia) and PCR amplification using the following oligonucleotides:

KRT14_F
TTTGGATCCACCATGACCACCTGCAGCCGCCAG,
KRT14_R
AATAAGCTTTCAGTTCTTGGTGCGAAGGACCTGC

and commercially available kit Phusion® High-Fidelity DNA Polymerase (New England Biolabs, USA); amplification product and DNA vector VTvaf17 were cleaved by restriction endonucleases BamHI and HindIII (New England Biolabs, USA).

This resulted in a 4575 bp DNA vector VTvaf17-KRT14 with the nucleotide sequence SEQ ID No. 2 and general structure shown in FIG. 1B.

Gene therapy DNA vector VTvaf17 was constructed as described in Example 1.

### Example 3

Production of gene therapy DNA vector VTvaf17-LAMB3 carrying the therapeutic gene, namely the human LAMB3 gene.

Gene therapy DNA vector VTvaf17-LAMB3 was constructed by cloning the coding region of LAMB3 gene (3521 bp) to a 3165 bp DNA vector VTvaf17 by SalI and EcoRI restriction sites. The coding region of LAMB3 gene (3521 bp) was obtained by isolating total RNA from the biological human tissue sample followed by reverse transcription reaction using commercial kit Mint-2 (Evrogen, Russia) and PCR amplification using the following oligonucleotides:

LAMB3_F
TTAGTCGACCACCATGAGACCATTCTTCCTCTTG,
LAMB3_R
ATAGAATTCACTTGCAGGTGGCATAGTAGAG

and commercially available kit Phusion® High-Fidelity DNA Polymerase (New England Biolabs, USA); amplification product and DNA vector VTvaf17 were cleaved by restriction endonucleases SalI and EcoRI (New England Biolabs, USA).

This resulted in a 6674 bp DNA vector VTvaf17-LAMB3 with the nucleotide sequence SEQ ID No. 3 and general structure shown in FIG. 1C.

Gene therapy DNA vector VTvaf17 was constructed as described in Example 1.

### Example 4

Production of gene therapy DNA vector VTvaf17-COL7A1 carrying the therapeutic gene, namely the COL7A1 gene.

Gene therapy DNA vector VTvaf17-COL7A1 was constructed by cloning the coding region of COL7A1 gene (8838 bp) to a 3165 bp DNA vector VTvaf17 by SalI and EcoRI restriction sites. The coding region of COL7A1 gene (8838 bp) was obtained by isolating total RNA from the biological human tissue sample followed by reverse transcription reaction using commercial kit Mint-2 (Evrogen) and PCR amplification using the following oligonucleotides:

COL7A1_F
ATCGTCGACCACCATGACGCTGCGGCTTCTGGT,
COL7A1_R
ATAGAATTCAGTCCTGGGCAGTACCTGTC

and commercially available kit Phusion® High-Fidelity DNA Polymerase (New England Biolabs, USA); amplification product and DNA vector VTvaf17 were cleaved by restriction endonucleases SalI and EcoRI (New England Biolabs, USA).

This resulted in a 11990 bp DNA vector VTvaf17-COL7A1 with the nucleotide sequence SEQ ID No. 4 and general structure shown in FIG. 1D.

Gene therapy DNA vector VTvaf17 was constructed as described in Example 1.

### Example 5

Proof of the ability of gene therapy DNA vector VTvaf17-KRT5 carrying the therapeutic gene, namely KRT5 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the KRT5 therapeutic gene were assessed in HDFa primary human dermal fibroblast cell culture (ATCC PCS-201-01) 48 hours after its transfection with gene therapy DNA vector VTvaf17-KRT5 carrying the human KRT5 gene. The amount of mRNA was determined by the dynamics of accumulation of cDNA amplicons in the real-time PCR.

HDFa primary human dermal fibroblast cell culture was used for the assessment of changes in the therapeutic KRT5 mRNA accumulation. HDFa cell culture was grown under standard conditions (37° C., 5% CO2) using the Fibroblast Growth Kit-Serum-Free (ATCC® PCS-201-040). The growth medium was replaced every 48 hours during the cultivation process.

To achieve 90% confluence, 24 hours before the transfection procedure, the cells were seeded into a 24-well plate in the quantity of 5×10⁴ cells per well. Transfection with gene therapy DNA vector VTvaf17-KRT5 expressing the human KRT5 gene was performed using Lipofectamine 3000 (ThermoFisher Scientific, USA) according to the manufacturer's recommendations. In test tube 1, 1 μl of DNA vector VTvaf17-KRT5 solution (concentration 500 ng/μl) and 1 μl of reagent P3000 was added to 25 μl of medium Opti-MEM (Gibco, USA). The preparation was mixed by gentle shaking. In test tube 2, 1 μl of Lipofectamine 3000 solution was added to 25 μl of medium Opti-MEM (Gibco, USA). The preparation was mixed by gentle shaking. The contents from test tube 1 were added to the contents of test tube 2, and the mixture was incubated at room temperature for 5 minutes. The resulting solution was added dropwise to the cells in the volume of 40 μl.

HDFa cells transfected with the gene therapy DNA vector VTvaf17 devoid of the inserted therapeutic gene (cDNA of KRT5 gene before and after transfection with gene therapy DNA vector VTvaf17 devoid of the inserted therapeutic gene is not shown in the figures) were used as a reference. Reference vector VTvaf17 for transfection was prepared as described above.

Total RNA from HDFa cells was extracted using Trizol Reagent (Invitrogen, USA) according to the manufacturer's recommendations. 1 ml of Trizol Reagent was added to the well with cells and homogenised and heated for 5 minutes at 65° C. Then the sample was centrifuged at 14,000 g for 10 minutes and heated again for 10 minutes at 65° C. Then 200 μl of chloroform was added, and the mixture was gently stirred and centrifuged at 14,000 g for 10 minutes. Then the water phase was isolated and mixed with 1/10 of the volume of 3M sodium acetate, pH 5.2, and an equal volume of isopropyl alcohol. The sample was incubated at −20° C. for 10 minutes and then centrifuged at 14,000 g for 10 minutes. The precipitated RNA were rinsed in 1 ml of 70% ethyl alcohol, air-dried and dissolved in 10 μl of RNase-free water. The level of KRT5 mRNA expression after transfection was determined by assessing the dynamics of the accumulation of cDNA amplicons by real-time PCR. For the production and amplification of cDNA specific for the human KRT5 gene, the following KRT5_SF and KRT5_SR oligonucleotides were used:

KRT5_SF
CGAAGCCGAGTCCTGGTATC,
KRT5_SR
TTGGCGCACTGTTTCTTGAC

The length of amplification product is 162 bp.

Reverse transcription reaction and PCR amplification was performed using SYBR GreenQuantitect RT-PCR Kit (Qiagen, USA) for real-time PCR. The reaction was carried out in a volume of 20 μl, containing: 25 μl of QuantiTect SYBR Green RT-PCR Master Mix, 2.5 mM of magnesium chloride, 0.5 uM of each primer, and 5 μl of RNA. For the reaction, CFX96 amplifier (Bio-Rad, USA) was used under the following conditions: 1 cycle of reverse transcription at 42° C. for 30 minutes, denaturation at 98° C. for 15 minutes, followed by 40 cycles comprising denaturation at 94° C. for 15s, annealing of primers at 60° C. for 30s and elongation at 72° C. for 30s. B2M (human beta-2-microglobuline) gene listed in the GenBank database under number NM 004048.2 was used as a reference gene. Positive control included amplicons from PCR on matrices represented by plasmids in known concentrations containing cDNA sequences of KRT5 and B2M genes. Negative control included deionised water. Real-time quantification of the dynamics of accumulation of cDNA amplicons of KRT5 and B2M genes was conducted using the Bio-Rad CFX Manager 2.1 software (Bio-Rad, USA). Diagrams resulting from the assay are shown in FIG. 2.

FIG. 2 shows that the level of specific mRNA of human KRT5 gene has grown massively as a result of transfection of HDFa primary human fibroblast cell culture with gene therapy DNA vector VTvaf17-KRT5, which confirms the ability of the vector to penetrate eukaryotic cells and express the KRT5 gene at the mRNA level. The presented results also confirm the practicability of use of gene therapy DNA vector VTvaf17-KRT5 in order to increase the expression level of KRT5 gene in eukaryotic cells.

### Example 6

Proof of the ability of gene therapy DNA vector VTvaf17-KRT14 carrying the therapeutic gene, namely KRT14 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the KRT14 therapeutic gene were assessed in HEKa primary human epidermal keratinocyte cell culture (ATCC PCS-200-011) 48 hours after its transfection with gene therapy DNA vector VTvaf17-KRT14 carrying the human KRT14 gene. The amount of mRNA was determined by the dynamics of accumulation of cDNA amplicons in the real-time PCR.

HEKa primary human epidermal keratinocyte cell culture was grown in Keratinocyte Growth Kit (ATCC® PCS-200-040™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-KRT14 expressing the human KRT14 gene was performed according to the procedure described in Example 5. B2M (beta-2-microglobuline) gene listed in the GenBank database under number NM 004048.2 was used as a reference gene. HEKa cell culture transfected with the gene therapy DNA vector VTvaf17 devoid of the therapeutic gene (cDNA of KRT14 gene before and after transfection with gene therapy DNA vector VTvaf17 devoid of the inserted therapeutic gene is not shown in the figure) was used as a reference. RNA isolation, reverse transcription reaction, and real-time PCR were performed as described in Example 5, except for oligonucleotides with sequences different from Example 5. For the amplification of cDNA specific for the human KRT14 gene, the following KRT14_SF and KRT14_SR oligonucleotides were used:

KRT14_SF
TCCAGGAGATGATTGGCAGC,
KRT14_SR
GGATGACTGCGATCCAGAGG

The length of amplification product is 197 bp.

Positive control included amplicons from PCR on matrices represented by plasmids in known concentrations containing cDNA sequences of KRT14 and B2M genes. Negative control included deionised water. Real-time quantification of the PCR products, i.e. KRT14 and B2M gene cDNAs obtained by amplification, was conducted using the Bio-Rad CFX Manager 2.1 software (Bio-Rad, USA). Diagrams resulting from the assay are shown in FIG. 3.

FIG. 3 shows that the level of specific mRNA of human KRT14 gene has grown massively as a result of transfection of HEKa cell culture with gene therapy DNA vector VTvaf17-KRT14, which confirms the ability of the vector to penetrate eukaryotic cells and express the KRT14 gene at the mRNA level. The presented results also confirm the practicability of use of gene therapy DNA vector VTvaf17-KRT14 in order to increase the expression level of KRT14 gene in eukaryotic cells.

### Example 7

Proof of the ability of gene therapy DNA vector VTvaf17-LAMB3 carrying the therapeutic gene, namely LAMB3 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the LAMB3 therapeutic gene were assessed in Human Skeletal Myoblasts (HSKM) (GIBCO® Cat. A12555) 48 hours after their transfection with gene therapy DNA-vector VTvaf17-LAMB3 carrying the human LAMB3 gene. The amount of mRNA was determined by the dynamics of accumulation of cDNA amplicons in the real-time PCR.

HSKM Human skeletal myoblast cell culture was grown in Dulbecco's Modified Eagle's Medium (DMEM) (ATCC® 30-2002™) with the addition of 2% of horse serum (Gibco Cat. 16050130) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-LAMB3 expressing the human LAMB3 gene was performed according to the procedure described in Example 5. B2M (beta-2-microglobuline) gene listed in the GenBank database under number NM 004048.2 was used as a reference gene. HSKM cell culture transfected with the gene therapy DNA vector VTvaf17 devoid of the therapeutic gene (cDNA of LAMB3 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 5, except for oligonucleotides with sequences different from Example 5. For the amplification of cDNA specific for the human LAMB3 gene, the following LAMB3_SF and LAMB3_SR oligonucleotides were used:

LAMB3_SF
CAGAGGAGCTGTTTGGGGAG,
LAMB3_SR
CCCATTGATGTGGTCACGGA

The length of amplification product is 155 bp.

Positive control included amplicons from PCR on matrices represented by plasmids in known concentrations containing cDNA sequences of LAMB3 and B2M genes. Negative control included deionised water. Real-time quantification of the PCR products, i.e. LAMB3 and B2M gene cDNAs obtained by amplification, was conducted using the Bio-Rad CFX Manager 2.1 software (Bio-Rad, USA). Diagrams resulting from the assay are shown in FIG. 4.

FIG. 4 shows that the level of specific mRNA of human LAMB3 gene has grown massively as a result of transfection of HSKM human skeletal myoblast cell culture with gene therapy DNA vector VTvaf17-LAMB3, which confirms the ability of the vector to penetrate eukaryotic cells and express the LAMB3 gene at the mRNA level. The presented results also confirm the practicability of use of gene therapy DNA vector VTvaf17-LAMB3 in order to increase the expression level of LAMB3 gene in eukaryotic cells.

### Example 8

Proof of the ability of gene therapy DNA vector VTvaf17-COL7A1 carrying the therapeutic gene, namely COL7A1 gene, to penetrate eukaryotic cells and its functional activity at the level of therapeutic gene mRNA expression. This example also demonstrates practicability of use of gene therapy DNA vector carrying the therapeutic gene.

Changes in the mRNA accumulation of the COL7A1 therapeutic gene were assessed in Primary Umbilical Vein Endothelial Cells; Normal, Human (HUVEC) (ATCC® PCS-100-010™) 48 hours after their transfection with gene therapy DNA-vector VTvaf17-COL7A1 carrying the human COL7A1 gene. The amount of mRNA was determined by the dynamics of accumulation of cDNA amplicons in the real-time PCR.

HUVEC human endothelial cell culture was grown in Vascular Cell Basal Medium (ATCC PCS-100-030) with the addition of Endothelial Cell Growth Kit-BBE (ATCC® PCS-100-040™) under standard conditions (37° C., 5% CO2). To achieve 90% confluence, 24 hours before the transfection procedure, the cells were seeded into a 24-well plate in the quantity of 5×10⁴ cells per well. Lipofectamine 3000 (ThermoFisher Scientific, USA) was used as a transfection reagent. The transfection with gene therapy DNA vector VTvaf17-COL7A1 expressing the human COL7A1 gene was performed according to the procedure described in Example 5. HUVEC human endothelial cell culture transfected with the gene therapy DNA vector VTvaf17 not carrying the therapeutic gene (cDNA of COL7A1 gene before and after transfection with gene therapy DNA vector VTvaf17 devoid of the inserted therapeutic gene is not shown in the figures) was used as a reference. RNA isolation, reverse transcription reaction, and real-time PCR were performed as described in Example 5, except for oligonucleotides with sequences different from Example 5. For the amplification of cDNA specific for the human COL7A1 gene, the following COL7A1_SF and COL7A1 SR oligonucleotides were used:

COL7A1_SF
CAAAGGAGAGATGGGGGAGC,
COL7A1_SR
ATCATTTCCACTGGGGCCTG

The length of amplification product is 184 bp.

Positive control included amplicons from PCR on matrices represented by plasmids in known concentrations containing cDNA sequences of COL7A1 and B2M genes. B2M (beta-2-microglobuline) gene listed in the GenBank database under number NM 004048.2 was used as a reference gene. Negative control included deionised water. Real-time quantification of the PCR products, i.e. COL7A1 and B2M gene cDNAs obtained by amplification, was conducted using the Bio-Rad CFX Manager 2.1 software (Bio-Rad, USA). Diagrams resulting from the assay are shown in FIG. 5.

FIG. 5 shows that the level of specific mRNA of human COL7A1 gene has grown massively as a result of transfection of HUVEC human endothelial cell culture with gene therapy DNA vector VTvaf17-COL7A1, which confirms the ability of the vector to penetrate eukaryotic cells and express the COL7A1 gene at the mRNA level. The presented results also confirm the practicability of use of gene therapy DNA vector VTvaf17-COL7A1 in order to increase the expression level of COL7A1 gene in eukaryotic cells.

### Example 9

Proof of the efficiency and practicability of use of gene therapy DNA vector VTvaf17-KRT5 carrying the KRT5 gene in order to increase the expression of KRT5 protein in mammalian cells.

The change in the KRT5 protein concentration in the lysate of HDFa human dermal fibroblast (ATCC PCS-201-01) was assessed after transfection of these cells with DNA vector VTvaf17-KRT5 carrying the human KRT5 gene.

Human dermal fibroblast cell culture was grown as described in Example 5.

To achieve 90% confluence, 24 hours before the transfection procedure, the cells were seeded into a 24-well plate in the quantity of 5×10⁴ cells per well. The 6th generation SuperFect Transfection Reagent (Qiagen, Germany) was used for transfection. The aqueous dendrimer solution without DNA vector (A) and DNA vector VTvaf17 devoid of cDNA of KRT5 gene (B) were used as a reference, and DNA vector VTvaf17-KRT5 carrying the human KRT5 gene (C) was used as the transfected agent. The DNA-dendrimer complex was prepared according to the manufacturer's procedure (QIAGEN, SuperFect Transfection Reagent Handbook, 2002) with some modifications. For cell transfection in one well of a 24-well plate, the culture medium was added to 1 μg of DNA vector dissolved in TE buffer to a final volume of 60 μl, then 5 μl of SuperFect Transfection Reagent was added and gently mixed by pipetting five times. The complex was incubated at room temperature for 10-15 minutes. Then the culture medium was taken from the wells, the wells were rinsed with 1 ml of PBS buffer. 350 μl of medium containing 10 μg/ml of gentamicin was added to the resulting complex, mixed gently, and added to the cells. The cells were incubated with the complexes for 2-3 hours at 37° C. in the presence of 5% CO2.

The medium was then removed carefully, and the live cell array was rinsed with 1 ml of PBS buffer. Then, medium containing 10 μg/ml of gentamicin was added and incubated for 24-48 hours at 37° C. in the presence of 5% CO2.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The KRT5 protein was assayed by enzyme-linked immunosorbent assay (ELISA) using the Human KRT5/CK5/Cytokeratin 5 ELISA Kit (Sandwich ELISA) (LifeSpan BioSciences Cat. LS-F8194-1, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of KRT5 protein was used. The sensitivity was at least 15.6 pg/ml, measurement range—from 15.6 pg/ml to 1000 pg/ml. R-3.0.2 was used for the statistical treatment of the results and data visualization (https://www.r-project.org/). Diagrams resulting from the assay are shown in FIG. 6.

FIG. 6 shows that the transfection of HDFa human dermal fibroblast cells with gene therapy DNA vector VTvaf17-KRT5 results in increased KRT5 protein concentration compared to reference samples, which confirms the ability of the vector to penetrate eukaryotic cells and express the KRT5 gene at the protein level. The presented results also confirm the practicability of use of gene therapy DNA vector VTvaf17-KRT5 in order to increase the expression level of KRT5 gene in eukaryotic cells.

### Example 10

Proof of the efficiency and practicability of use of gene therapy DNA vector VTvaf17-KRT14 carrying the KRT14 gene in order to increase the expression of KRT14 protein in mammalian cells.

The change in the KRT14 protein concentration in the cell lysate of HEKa primary human epidermal keratinocyte cell culture (ATCC PCS-200-01) was assessed after transfection of these cells with the DNA vector VTvaf17-KRT14 carrying the human KRT14 gene. Cells were grown as described in Example 6.

The 6th generation SuperFect Transfection Reagent (Qiagen, Germany) was used for transfection. The aqueous dendrimer solution without DNA vector (A) and DNA vector VTvaf17 devoid of cDNA of KRT14 gene (B) were used as a reference, and DNA vector VTvaf17-KRT14 carrying the human KRT14 gene (C) was used as the transfected agent. Preparation of the DNA dendrimer complex and transfection of HEKa cells were performed according to the procedure described in Example 9.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein. The KRT14 protein was assayed by enzyme-linked immunosorbent assay (ELISA) using the Human KRT14/CK14/Cytokeratin 14 ELISA Kit (Sandwich ELISA) (LifeSpan BioSciences Cat. LS-F7936, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of KRT14 protein was used. The sensitivity was at least 156 pg/ml, measurement range—from 156 pg/ml to 10000 pg/ml. R-3.0.2 was used for the statistical treatment of the results and data visualization (https://www.r-project.org/). Diagrams resulting from the assay are shown in FIG. 7.

FIG. 7 shows that the transfection of HEKa primary human epidermal keratinocyte cell culture with gene therapy DNA vector VTvaf17-KRT14 results in increased KRT14 protein concentration compared to reference samples, which confirms the ability of the vector to penetrate eukaryotic cells and express the KRT14 gene at the protein level. The presented results also confirm the practicability of use of gene therapy DNA vector VTvaf17-KRT14 in order to increase the expression level of KRT14 gene in eukaryotic cells.

### Example 11

Proof of the efficiency and practicability of use of gene therapy DNA vector VTvaf17-LAMB3 carrying the LAMB3 gene in order to increase the expression of LAMB3 protein in mammalian cells.

The change in the LAMB3 protein concentration in the lysate of Human Skeletal Myoblasts (HSKM) (GIBCOR Cat. A12555) was assessed after transfection of these cells with DNA vector VTvaf17-LAMB3 carrying the human LAMB3 gene. Cells were cultured as described in Example 7.

The 6th generation SuperFect Transfection Reagent (Qiagen, Germany) was used for transfection. The aqueous dendrimer solution without DNA vector (A) and DNA vector VTvaf17 devoid of cDNA of LAMB3 gene (B) were used as a reference, and DNA vectorVTvaf17-LAMB3 carrying the human LAMB3 gene (C) was used as the transfected agent. Preparation of the DNA dendrimer complex and transfection of HSKM cells were performed according to the procedure described in Example 9.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The LAMB3 protein was assayed enzyme-linked immunosorbent assay (ELISA) using the Human LAMB3/Laminin Beta 3 ELISA Kit (Sandwich ELISA) (LifeSpan BioSciences Cat. LS-F33141, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of LAMB3 protein was used. The sensitivity was at least 9.375 pg/ml, measurement range—from 15.625 pg/ml to 8000 pg/ml. R-3.0.2 was used for the statistical treatment of the results and data visualization (https://www.r-project.org/). Diagrams resulting from the assay are shown in FIG. 8.

FIG. 8 shows that the transfection of HSKM human skeletal myoblast cell culture with gene therapy DNA vector VTvaf17-LAMB3 results in increased LAMB3 protein concentration compared to reference samples, which confirms the ability of the vector to penetrate eukaryotic cells and express the LAMB3 gene at the protein level. The presented results also confirm the practicability of use of gene therapy DNA vector VTvaf17-LAMB3 in order to increase the expression level of LAMB3 gene in eukaryotic cells.

### Example 12

Proof of the efficiency and practicability of use of gene therapy DNA vector VTvaf17-COL7A1 carrying the COL7A1 gene in order to increase the expression of COL7A1 protein in mammalian cells.

The change in the COL7A1 protein concentration in the cell lysate of Primary Umbilical Vein Endothelial Cells; Normal, Human (HUVEC) (ATCC® PCS-100-010™) was assessed 48 hours after transfection of these cells with gene therapy DNA-vector VTvaf17-COL7A1 carrying the human COL7A1 gene. Cells were cultured as described in Example 8.

The 6th generation SuperFect Transfection Reagent (Qiagen, Germany) was used for transfection. The aqueous dendrimer solution without DNA vector (A) and DNA vector VTvaf17 devoid of cDNA of COL7A1 gene (B) were used as a reference, and DNA vector VTvaf17-COL7A1 carrying the human COL7A1 gene (C) was used as the transfected agent. Preparation of the DNA dendrimer complex and transfection of HUVEC cells were performed according to the procedure described in Example 9.

After transfection, cells were rinsed three times with PBS, and then 1 ml of PBS was added to the cells and the cells were subjected to freezing/thawing three times. Then the suspension was centrifuged for 15 minutes at 15,000 rpm, and supernatant was collected and used for the quantification and assay of the therapeutic protein.

The COL7A1 protein was assayed by enzyme-linked immunosorbent assay (ELISA) using the Human COL7A1/Collagen VII ELISA Kit (Sandwich ELISA) (LifeSpan BioSciences Cat. LS-F11164, USA) according to the manufacturer's method with optical density detection using ChemWell Automated EIA and Chemistry Analyser (Awareness Technology Inc., USA).

To measure the numerical value of concentration, the calibration curve constructed using the reference samples from the kit with known concentrations of COL7A1 protein was used. The sensitivity was at least 156 pg/ml, measurement range—from 156 pg/ml to 10000 pg/ml. R-3.0.2 was used for the statistical treatment of the results and data visualization (https://www.r-project.org/). Diagrams resulting from the assay are shown in FIG. 9.

FIG. 9 shows that the transfection of HUVEC primary human endothelial cell culture with gene therapy DNA vector VTvaf17-COL7A1 results in increased COL7A1 protein concentration compared to reference samples, which confirms the ability of the vector to penetrate eukaryotic cells and express the COL7A1 gene at the protein level. The presented results also confirm the practicability of use of gene therapy DNA vector VTvaf17-COL7A1 in order to increase the expression level of the COL7A1 gene in eukaryotic cells.

### Example 13

Proof of the efficiency and practicability of use of gene therapy DNA vector VTvaf17-COL7A1 carrying the COL7A1 gene in order to increase the expression of COL7A1 protein in human tissues.

To prove the efficiency of gene therapy DNA vector VTvaf17-COL7A1 carrying the therapeutic gene, namely the COL7A1 gene, and practicability of its use, changes in COL7A1 protein concentration in human skin upon injection of gene therapy DNA vector VTvaf17-COL7A1 carrying the human COL7A1 gene were assessed.

To analyse changes in the COL7A1 protein concentration, gene therapy DNA vector VTvaf17-COL7A1 carrying the COL7A1 gene was injected into the forearm skin of three patients with concurrent injection of a placebo being gene therapy DNA vector VTvaf17 devoid of cDNA of COL7A1 gene.

Patient 1, woman, 44 y.o. (P1); Patient 2, woman, 50 y.o. (P2); Patient 3, man, 53 y.o. (P3). Polyethyleneimine Transfection reagent cGMP grade in-vivo-jetPEI (Polyplus Transfection, France) was used as a transport system. Gene therapy DNA vector VTvaf17-COL7A1 containing cDNA of COL7A1 gene and gene therapy DNA vector VTvaf17 used as a placebo not containing cDNA of COL7A1 gene were dissolved in sterile nuclease-free water. To obtain a gene construct, DNA-cGMP grade in-vivo-jetPEI complexes were prepared according to the manufacturer recommendations.

Gene therapy DNA vector VTvaf17 (placebo) and gene therapy DNA vector VTvaf17-COL7A1 carrying the COL7A1 gene were injected in the quantity of 1 mg for each genetic construct using the tunnel method with a 30G needle to the depth of 2-3 mm. The injectate volume of gene therapy DNA vector VTvaf17 (placebo) and gene therapy DNA vector VTvaf17-COL7A1 carrying the COL7A1 gene was 0.3 ml for each genetic construct. The points of injection of each genetic construct were located at 8 to 10 cm intervals at the forearm site.

The biopsy samples were taken on the 2nd day after the injection of the genetic constructs of gene therapy DNA vectors. The biopsy samples were taken from the patients' skin in the site of injection of gene therapy DNA vector VTvaf17-COL7A1 carrying the COL7A1 gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used in order to assay the therapeutic protein by enzyme-linked immunosorbent assay (ELISA). The COL7A1 protein was performed by enzyme-linked immunosorbent assay (ELISA) as described in Example 12 with optical density detection using ChemWell Automated EIA and Chemistry Analyser (Awareness Technology Inc., USA).

R-3.0.2 was used for the statistical treatment of the results and data visualization (https://www.r-project.org/) according to the manufacturer's method with optical density detection using ChemWell Automated EIA and Chemistry Analyser (Awareness Technology Inc., USA). Diagrams resulting from the assay are shown in FIG. 10.

FIG. 10 shows the increased COL7A1 protein concentration in the skin of all three patients in the injection site of gene therapy DNA vector VTvaf17-COL7A1 carrying the human COL7A1 therapeutic gene compared to the COL7A1 protein concentration in the injection site of gene therapy DNA vector VTvaf17 (placebo) devoid of the human COL7A1 gene, which indicates the efficiency of gene therapy DNA vector VTvaf17-COL7A1 and confirms the practicability of its use, in particular upon intracutaneous injection of gene therapy DNA vector in human tissues.

### Example 14

Proof of the efficiency and practicability of use of gene therapy DNA vector VTvaf17-LAMB3 carrying the LAMB3 gene in order to increase the expression of LAMB3 protein in human tissues.

To confirm the efficiency of gene therapy DNA vector VTvaf17-LAMB3 carrying the LAMB3 therapeutic gene and practicability of its use, the change in the LAMB3 protein concentration in human muscle tissues upon injection of gene therapy DNA vector VTvaf17-LAMB3 carrying the therapeutic gene, namely the human LAMB3 gene, was assessed.

To analyse changes in the concentration of LAMB3 protein, gene therapy DNA vector VTvaf17-LAMB3 carrying the LAMB3 gene with transport molecule was injected into the gastrocnemius muscle of three patients with concurrent injection of a placebo being gene therapy DNA vector VTvaf17 devoid of cDNA of LAMB3 gene with transport molecule.

Patient 1, woman, 40 y.o. (P1); Patient 2, woman, 43 y.o. (P2); Patient 3, man, 54 y.o. (P3). Polyethyleneimine Transfection reagent cGMP grade in-vivo-jetPEI (Polyplus Transfection, France) was used as a transport system; sample preparation was carried out in accordance with the manufacturer's recommendations.

Gene therapy DNA vector VTvaf17 (placebo) and gene therapy DNA vector VTvaf17-LAMB3 carrying the LAMB3 gene were injected in the quantity of 1 mg for each genetic construct using the tunnel method with a 30G needle to the depth of around 10 mm. The injectate volume of gene therapy DNA vector VTvaf17 (placebo) and gene therapy DNA vector VTvaf17-LAMB3 carrying the LAMB3 gene was 0.3 ml for each genetic construct. The points of injection of each genetic construct were located medially at 8 to 10 cm intervals.

The biopsy samples were taken on the 2nd day after the injection of the genetic constructs of gene therapy DNA vectors. The biopsy samples were taken from the patients' muscle tissues in the site of injection of gene therapy DNA vector VTvaf17-LAMB3 carrying the LAMB3 gene (I), gene therapy DNA vector VTvaf17 (placebo) (II), and intact site of gastrocnemius muscle (III) using the skin biopsy device MAGNUM (BARD, USA). The skin of patients in the biopsy site was preliminarily rinsed with sterile saline and anaesthetised with a lidocaine solution. The biopsy sample size was ca. 20 mm3, and the weight was up to 22 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein.

The LAMB3 protein was assayed by enzyme-linked immunosorbent assay (ELISA) as described in Example 11 with optical density detection using ChemWell Automated EIA and Chemistry Analyser (Awareness Technology Inc., USA).

R-3.0.2 was used for the statistical treatment of the results and data visualisation (https://www.r-project.org/). Diagrams resulting from the assay are shown in FIG. 11.

FIG. 11 shows the increased LAMB3 protein concentration in the gastrocnemius muscle of all three patients in the injection site of gene therapy DNA vector VTvaf17-LAMB3 carrying the therapeutic gene, namely the human LAMB3 gene, compared to the LAMB3 protein concentration in the injection site of gene therapy DNA vector VTvaf17 (placebo) devoid of the human LAMB3 gene, which indicates the efficiency of gene therapy DNA vector VTvaf17-LAMB3 and confirms the practicability of its use, in particular upon intramuscular injection of gene therapy DNA vector in human tissues.

### Example 15

Proof of the efficiency and practicability of use of gene therapy DNA vector VTvaf17-KRT14 carrying the KRT14 gene in order to increase the expression of KRT14 protein in human tissues.

To confirm the efficiency of gene therapy DNA vector VTvaf17-KRT14 carrying the therapeutic gene, namely the KRT14 gene, and practicability of its use, changes in KRT14 protein concentration in human skin upon injection of gene therapy DNA vector VTvaf17-KRT14 carrying the human KRT14 gene were assessed.

To analyse changes in the KRT14 protein concentration, gene therapy DNA vector VTvaf17-KRT14 carrying the KRT14 gene was injected into the forearm skin of three patients with concurrent injection of a placebo being gene therapy DNA vector VTvaf17 devoid of cDNA of KRT14 gene.

Patient 1, woman, 39 y.o. (P1); Patient 2, man, 61 y.o. (P2); Patient 3, man, 38 y.o. (P3). Polyethyleneimine Transfection reagent cGMP grade in-vivo-jetPEI (Polyplus Transfection, France) was used as a transport system. Gene therapy DNA vector VTvaf17-KRT14 containing cDNA of KRT14 gene and gene therapy DNA vector VTvaf17 used as a placebo not containing cDNA of KRT14 gene were dissolved in sterile nuclease-free water. To obtain a gene construct, DNA-cGMP grade in-vivo-jetPEI complexes were prepared according to the manufacturer recommendations.

Gene therapy DNA vector VTvaf17 (placebo) and gene therapy DNA vector VTvaf17-KRT14 carrying the KRT14 gene were injected in the quantity of 1 mg for each genetic construct using the tunnel method with a 30G needle to the depth of 1.5-2 mm. The injectate volume of gene therapy DNA vector VTvaf17 (placebo) and gene therapy DNA vector VTvaf17-KRT14 carrying the KRT14 gene was 0.3 ml for each genetic construct. The points of injection of each genetic construct were located at 8 to 10 cm intervals at the forearm site.

The biopsy samples were taken on the 2nd day after the injection of the genetic constructs of gene therapy DNA vectors. The biopsy samples were taken from the patients' skin in the site of injection of gene therapy DNA vector VTvaf17-KRT14 carrying the KRT14 gene (I), gene therapy DNA vector VTvaf17 (placebo) (II), and from intact skin (III) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic angiogenin protein by enzyme-linked immunosorbent assay (ELISA) as described in Example 10 with optical density detection using ChemWell Automated EIA and Chemistry Analyser (Awareness Technology Inc., USA).

R-3.0.2 was used for the statistical treatment of the results and data visualization (https://www.r-project.org/). Diagrams resulting from the assay are shown in FIG. 12.

FIG. 12 shows the increased KRT14 protein concentration in the skin of all three patients in the injection site of gene therapy DNA vector VTvaf17-KRT14 carrying the human KRT14 therapeutic gene compared to the KRT14 protein concentration in the injection site of gene therapy DNA vector VTvaf17 (placebo) devoid of the human KRT14 gene, which indicates the efficiency of gene therapy DNA vector VTvaf17-KRT14 and confirms the practicability of its use, in particular upon intracutaneous injection of gene therapy DNA vector in human tissues.

### Example 16

Proof of the efficiency of gene therapy DNA vector VTvaf17-KRT14 carrying the KRT14 gene and practicability of its use in order to increase the expression level of the KRT14 protein in human tissues by injecting autologous fibroblasts transfected with gene therapy DNA vector VTvaf17-KRT14.

To confirm the efficiency of gene therapy DNA vector VTvaf17-KRT14 carrying the KRT14 gene and practicability of its use, changes in the KRT14 protein concentration in human skin upon injection of patient's skin with autologous fibroblast culture of the same patient transfected with gene therapy DNA vector VTvaf17-KRT14 were assessed.

The appropriate autologous fibroblast culture transfected with the gene therapy DNA vector VTvaf17-KRT14 carrying the KRT14 gene was injected into the patient's forearm skin with concurrent injection of a placebo in the form of autologous fibroblast culture transfected with gene therapy DNA vector VTvaf17 not carrying the KRT14 gene.

The human primary fibroblast culture was isolated from the patient skin biopsy specimens. Biopsy specimens of the skin from the area protected by ultraviolet, namely behind the ear or on the inner lateral side of the elbow, were taken using the skin biopsy device Epitheasy 3.5 (Medax SRL, Italy). The biopsy sample was ca. 10 mm and ca. 11 mg. The patient's skin was preliminarily rinsed with sterile saline and anaesthetised with a lidocaine solution. The primary cell culture was cultivated at 37° C. in the presence of 5% CO2, in the DMEM medium with 10% fetal bovine serum and 100U/ml of ampicillin. The passage and change of culture medium were performed every 2 days. Total duration of culture growth did not exceed 25-30 days. Then an aliquot of 5×10⁴ cells was taken from the cell culture. The patient's fibroblast culture was transfected with the gene therapy DNA vector VTvaf17-KRT14 carrying the KRT14 gene and placebo, i.e. VTvaf17 vector not carrying the KRT14 therapeutic gene.

The transfection was carried out using a cationic polymer such as polyethyleneimine JETPEI (Polyplus transfection, France), according to the manufacturer's instructions. The cells were cultured for 72 hours and then injected into the patient. Injection of autologous fibroblast culture of the patient transfected with gene therapy DNA vector VTvaf17-KRT14 and autologous fibroblast culture of the patient transfected with gene therapy DNA vector VTvaf17 as a placebo was performed in the forearm using the tunnel method with a 13 mm long 30G needle to the depth of approximately 3 mm. The concentration of the modified autologous fibroblasts in the injected suspension was approximately 5 mln cells per 1 ml of the suspension, the dose of the injected cells did not exceed 15 mln. The points of injection of the autologous fibroblast culture were located at 8 to 10 cm intervals.

Biopsy samples were taken on the 4th day after the injection of autologous fibroblast culture transfected with the gene therapy DNA vector VTvaf17-KRT14 carrying the therapeutic gene, namely KRT14 gene, and placebo. Biopsy was taken from the patient's skin in the site of injection of autologous fibroblast culture transfected with gene therapy DNA vector VTvaf17-KRT14 carrying the therapeutic gene, namely KRT14 gene (C), autologous fibroblast culture non-transfected with gene therapy DNA vector VTvaf17 not carrying the KRT14 therapeutic gene (placebo) (B), as well as from intact skin site (A) using the skin biopsy device Epitheasy 3.5 (Medax SRL, Italy). The skin of patients in the biopsy site was preliminarily rinsed with sterile saline and anaesthetised with a lidocaine solution. The biopsy sample size was ca. 10 mm3, and the weight was approximately 11 mg. The sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic protein as described in Example 15.

Diagrams resulting from the assay are shown in FIG. 13.

FIG. 13 shows the increased concentration of KRT14 protein in the area of the patient's skin in the injection site of autologous fibroblast culture transfected with the gene therapy DNA vector VTvaf17-KRT14 carrying the KRT14 gene compared to the KRT14 protein concentration in the injection site of autologous fibroblast culture transfected with the gene therapy DNA vector VTvaf17 that does not carry the KRT14 gene (placebo), which indicates the efficiency of gene therapy DNA vector VTvaf17-KRT14 and practicability of its use in order to increase the expression level of KRT14 in human tissues, in particular upon injection of autologous fibroblasts transfected with the gene therapy DNA vector VTvaf17-KRT14 into the skin.

### Example 17

Proof of the efficiency and practicability of combined use of gene therapy DNA vector VTvaf17-KRT5 carrying the KRT5 gene, gene therapy DNA vector VTvaf17-KRT14 carrying the KRT14 gene, gene therapy DNA vector VTvaf17-LAMB3 carrying the LAMB3 gene, and gene therapy DNA vector VTvaf17-COL7A1 carrying the COL7A1 gene for the increase of expression level of KRT5, KRT14, LAMB3, and COL7A1 proteins in mammalian tissues.

The changes in the KRT5, KRT14, LAMB3 and COL7A1 protein concentrations in the Wistar rat skin site were assessed upon injection of a mixture of gene therapy vectors into this site.

Polyethyleneimine Transfection reagent cGMP grade in-vivo-jetPEI (Polyplus Transfection, France) was used as a transport system. Equimolar mixture of four gene therapy DNA vectors, as well as gene therapy DNA vector VTvaf17 used as a placebo were dissolved in sterile nuclease-free water. To obtain a gene construct, DNA-cGMP grade in-vivo-jetPEI complexes were prepared according to the manufacturer recommendations. The injectate volume of each genetic construct (a mixture of gene therapy DNA vectors and placebo) was 0.3 ml with a total quantity of DNA equal to 100 μg. The solution injection was made into the rat skin using the insulin syringe to the depth of 1-1.5 mm.

The biopsy samples were taken on the 2nd day after the injection of the gene therapy DNA vectors. The biopsy sample was taken from the scar areas on the skin of animals in the injection site of a mixture of four gene therapy DNA vectors carrying the genes KRT5, KRT14, LAMB3, and COL7A1 (site I), gene therapy DNA vector VTvaf17 (placebo) (site II), as well as from the similar skin site not subjected to any manipulations (site III), using the skin biopsy device Epitheasy 3.5 (Medax SRL). The biopsy sample site was preliminarily rinsed with sterile saline and anaesthetised with a lidocaine solution. The biopsy sample size was ca. 10 mm3, and the weight was approximately 11 mg. Each sample was placed in a buffer solution containing 50 mM of Tris-HCl, pH 7.6, 100 mM of NaCl, 1 mM of EDTA, and 1 mM of phenylmethylsulfonyl fluoride, and homogenised to obtain a homogenised suspension. The suspension was then centrifuged for 10 minutes at 14,000 g. Supernatant was collected and used to assay the therapeutic proteins as described in Example 9 (quantification of KRT5 protein), Example 10 (quantification of KRT14 protein), Example 11 (quantification of LAMB3 protein), and Example 12 (quantification of COL7A1 protein). Diagrams resulting from the assay are shown in FIG. 14.

FIG. 14 demonstrates that there was an increase of KRT5, KRT14, LAMB3, and COL7A1 protein concentration in the rat skin site (site I) where a mixture of gene therapy DNA vector VTvaf17-KRT5 carrying the KRT5 therapeutic gene, therapy DNA vector VTvaf17-KRT14 carrying the KRT14 therapeutic gene, gene therapy DNA vector VTvaf17-LAMB3 carrying the LAMB3 therapeutic gene, gene therapy DNA vector VTvaf17-COL7A1 carrying the COL7A1 therapeutic gene were injected compared to site II (placebo site) and site III (intact site). The obtained results show the efficiency of combined use of gene therapy DNA vectors and practicability of use for the increase of the expression level of therapeutic proteins in mammalian tissues.

### Example 18

Proof of the efficiency of gene therapy DNA vector VTvaf17-LAMB3 carrying the LAMB3 gene and practicability of its use in order to increase the expression level of LAMB3 protein in mammalian cells.

To prove the efficiency of gene therapy DNA vector VTvaf17-LAMB3 carrying the LAMB3 gene, changes in mRNA accumulation of the LAMB3 therapeutic gene in MDBK bovine kidney epithelial cells (NBL-1) (ATCC® CCL-22™) 48 hours after their transfection with gene therapy DNA vector VTvaf17-LAMB3G carrying the human LAMB3 gene were assessed.

MDBK bovine kidney epithelial cells (NBL-1) were grown in Eagle's Minimum Essential Medium (EMEM) (ATCC® 30-2003™) with the addition of 10% Horse Serum (ATCC® 30-2040™). Transfection with gene therapy DNA vector VTvaf17-LAMB3 carrying the human LAMB3 gene and DNA vector VTvaf17 not carrying the human LAMB3 gene (reference), RNA extraction, reverse transcription reaction, PCR amplification, and data analysis were performed as described in Example 7. Bull/cow actin gene (ACT) listed in the GenBank database under number AH001130.2 was used as a reference gene. Positive control included amplicons from PCR on matrices represented by plasmids in known concentrations containing LAMB3 and ACT gene sequences. Negative control included deionised water. Real-time quantification of the PCR products, i.e. LAMB3 and ACT gene cDNAs obtained by amplification, was conducted using the Bio-Rad CFX Manager 2.1 software (Bio-Rad, USA).

Diagrams resulting from the assay are shown in FIG. 15.

FIG. 15 shows that the level of specific cDNA of human LAMB3 gene has grown massively as a result of transfection of MDBK bovine kidney epithelial cells with gene therapy DNA vector VTvaf17-LAMB3, which confirms the ability of the vector to penetrate eukaryotic cells and express the LAMB3 gene at the mRNA level. The presented results confirm the practicability of use of gene therapy DNA vector VTvaf17-LAMB3 in order to increase the expression level of LAMB3 gene in mammalian cells.

### Example 19

Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 carrying gene therapy DNA vector, method of production thereof.

The construction of strain for the production of gene therapy DNA vector based on gene therapy DNA vector VTvaf17 carrying the therapeutic gene on an industrial scale selected from the group of the following genes: KRT5, KRT14, LAMB3, and COL7A1, namely Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 carrying the gene therapy DNA vector VTvaf17-KRT5, or VTvaf17-KRT14, or VTvaf17-LAMB3, or VTvaf17-COL7A1, respectively, for its production allowing for antibiotic-free selection involves making electrocompetent cells of Escherichia coli strain SCS110-AF and subjecting these cells to electroporation with gene therapy DNA vector VTvaf17-KRT5, or DNA vector VTvaf17-KRT14, or DNA vector VTvaf17-LAMB3, or DNA vector VTvaf17-COL7A1. After that, the cells were poured into agar plates (Petri dishes) with a selective medium containing yeastrel, peptone, 6% sucrose, and 10 μg/ml of chloramphenicol. At the same time, production of Escherichia coli strain SCS110-AF for the production of gene therapy DNA vector VTvaf17 or gene therapy DNA vectors based on it allowing for antibiotic-free positive selection involves constructing a 64 bp linear DNA fragment that contains regulatory element RNA-IN of transposon Tn10 allowing for antibiotic-free positive selection, a 1422 bp levansucrase gene sacB, the product of which ensures selection within a sucrose-containing medium, a 763 bp chloramphenicol resistance gene catR required for the selection of strain clones in which homologous recombination occurs, and two homologous sequences, 329 bp and 233 bp, ensuring homologous recombination in the region of gene recA concurrent with gene inactivation, and then the Escherichia coli cells are transformed by electroporation, and clones surviving in a medium containing 10 μg/ml of chloramphenicol are selected.

The obtained strains for production were included in the collection of the National Biological Resource Centre—Russian National Collection of Industrial Microorganisms (NBRC RNCIM), RF and NCIMB Patent Deposit Service, UK under the following registration numbers:
Escherichia coli strain SCS110-AF/VTvaf17-KRT5—registered at the Russian National Collection of Industrial Microorganisms under number B-13386, date of deposit: 14 Dec. 2018; accession No. NCIMB: 43310, date of deposit: 13 Dec. 2018,
Escherichia coli strain SCS110-AF/VTvaf17-KRT14—registered at the Russian National Collection of Industrial Microorganisms under number B-13345, date of deposit: 22 Nov. 2018; accession No. NCIMB: 43282, date of deposit: 22 Nov. 2018.

### Example 20

The method for scaling up of the gene therapy DNA vector based on gene therapy DNA vector VTvaf17 carrying the therapeutic gene selected from the group of KRT5, KRT14, LAMB3, and COL7A1 to an industrial scale.

To confirm the producibility and constructability of gene therapy DNA vector VTvaf17-KRT5 (SEQ ID No. 1), or VTvaf17-KRT14 (SEQ ID No. 2), or VTvaf17-LAMB3 (SEQ ID No. 3), or VTvaf17-COL7A1 (SEQ ID No. 4) on an industrial scale, large-scale fermentation of Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 each containing gene therapy DNA vector VTvaf17 carrying the therapeutic gene, namely KRT5, or KRT14, or LAMB3, or COL7A1, was performed. Each Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 was produced based on Escherichia coli strain SCS110-AF (Cell and Gene Therapy LLC, United Kingdom) as described in Example 19 by electroporation of competent cells of this strain with the gene therapy DNA vector VTvaf17-KRT5, or VTvaf17-KRT14, or VTvaf17-LAMB3, or VTvaf17-COL7A1 carrying the therapeutic gene, namely KRT5, or KRT14, or LAMB3, or COL7A1 with further inoculation of transformed cells into agar plates (Petri dishes) with a selective medium containing yeastrel, peptone, and 6% sucrose, and selection of individual clones.

Fermentation of Escherichia coli SCS110-AF/VTvaf17-KRT5 carrying gene therapy DNA vector VTvaf17-KRT5 was performed in a 101 fermenter with subsequent extraction of gene therapy DNA vector VTvaf17-KRT5.

For the fermentation of Escherichia coli strain SCS110-AF/VTvaf17-KRT5, a medium was prepared containing (per 101 of volume): 100 g of tryptone and 50 g of yeastrel (Becton Dickinson, USA); then the medium was diluted with water to 8800 ml and autoclaved at 121° C. for 20 minutes, and then 1200 ml of 50% (w/v) sucrose was added. After that, the seed culture of Escherichia coli strain SCS110-AF/VTvaf17-KRT5 was inoculated into a culture flask in the volume of 100 ml. The culture was incubated in an incubator shaker for 16 hours at 30° C. The seed culture was transferred to the Techfors S bioreactor (Infors HT, Switzerland) and grown to a stationary phase. The process was controlled by measuring optical density of the culture at 600 nm. The cells were pelleted for 30 minutes at 5,000-10,000 g. Supernatant was removed, and the cell pellet was re-suspended in 10% (by volume) phosphate buffered saline. The cells were centrifuged again for 30 minutes at 5,000-10,000 g. Supernatant was removed, a solution of 20 mM TrisCl, 1 mM EDTA, 200 g/l sucrose, pH 8.0 was added to the cell pellet in the volume of 1000 ml, and the mixture was stirred thoroughly to a homogenised suspension. Then egg lysozyme solution was added to the final concentration of 100 μg/ml. The mixture was incubated for 20 minutes on ice while stirring gently. Then 2500 ml of 0.2M NaOH, 10 g/l sodium dodecyl sulphate (SDS) was added, the mixture was incubated for 10 minutes on ice while stirring gently, then 3500 ml of 3M sodium acetate, 2M acetic acid, pH 5-5.5 was added, and the mixture was incubated for 10 minutes on ice while stirring gently. The resulting sample was centrifuged for 20-30 minutes at 15,000 g or a greater value. The solution was decanted delicately, and residual precipitate was removed by passing through a coarse filter (filter paper). Then, RNase A (Sigma, USA) was added to the final concentration of 20 μg/ml, and the solution was incubated overnight for 16 hours at room temperature. The solution was then centrifuged for 20-30 minutes at 15,000 g and passed through a 0.45 μm membrane filter (Millipore, USA). Then, ultrafiltration was performed with a 100 kDa membrane (Millipore, USA) and the mixture was diluted to the initial volume with a buffer solution of 25 mM TrisCl, pH 7.0. This manipulation was performed three to four times. The solution was applied to the column with 250 ml of DEAE Sepharose HP (GE, USA), equilibrated with 25 mM TrisCl, pH 7.0. After the application of the sample, the column was washed with three volumes of the same solution and then gene therapy DNA vector VTvaf17-KRT5 was eluted using a linear gradient of 25 mM TrisCl, pH 7.0, to obtain a solution of 25 mM TrisCl, pH 7.0, 1M NaCl, five times the volume of the column. The elution process was controlled by measuring optical density of the run-off solution at 260 nm. Chromatographic fractions containing gene therapy DNA vector VTvaf17-KRT5 were joined together and subjected to gel filtration using Superdex 200 (GE, USA). The column was equilibrated with phosphate buffered saline. The elution process was controlled by measuring optical density of the run-off solution at 260 nm, and the fractions were analysed by agarose gel electrophoresis. The fractions containing gene therapy DNA vector VTvaf17-KRT5 were joined together and stored at −20° C. To assess the process reproducibility, the indicated processing operations were repeated five times. All processing operations for Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 were performed in a similar way.

The process reproducibility and quantitative characteristics of final product yield confirm the producibility and constructability of gene therapy DNA vector VTvaf17-KRT5, or VTvaf17-KRT14, or VTvaf17-LAMB3, or VTvaf17-COL7A1 on an industrial scale.

Thus, the produced gene therapy DNA vector containing the therapeutic gene can be used to deliver it to the cells of human beings and animals that experience reduced or insufficient expression of protein encoded by this gene, thus ensuring the desired therapeutic effect.

The purpose set in this invention, namely the construction of the gene therapy DNA vectors in order to increase the expression level of KRT5, KRT14, LAMB3, and COL7A1 genes that combine the following properties:
I) The effectiveness of increase of expression of therapeutic genes in eukaryotic cells due to the obtained gene therapy vectors with a vector part not exceeding 3200 bp;
II) Possibility of safe use in gene therapy of human beings and animals due to the absence of regulatory elements representing the nucleotide sequences of viral genomes and antibiotic resistance genes in the gene therapy DNA vector,
III) Producibility and constructability in the strains on an industrial scale,
IV)
as well as the purpose of the construction of strains carrying these gene therapy DNA vectors for the production of these gene therapy DNA vectors is achieved, which is supported by the following examples:
for Item I—Example 1, 2, 3, 4, 5; 6; 7; 8; 9; 10; 11; 12; 13; 14; 15; 16; 17; 18;
for Item II—Example 1, 2, 3, 4;
for Item III and Item IV—Example 19, 20.

### INDUSTRIAL APPLICABILITY

All the examples listed above confirm the industrial applicability of the proposed gene therapy DNA vector based on gene therapy DNA vector VTvaf17 carrying the therapeutic gene selected from the group of KRT5, KRT14, LAMB3, and COL7A1 genes in order to increase the expression level of these therapeutic genes, method of its production and use, Escherichia coli strain SCS110-AF/VTvaf17-KRT5, or Escherichia coli strain SCS110-AF/VTvaf17-KRT14, or Escherichia coli strain SCS110-AF/VTvaf17-LAMB3, or Escherichia coli strain SCS110-AF/VTvaf17-COL7A1 carrying gene therapy DNA vector, and method of its production on an industrial scale.

### List of Abbreviations

VTvaf17—Gene therapy vector devoid of sequences of viral genomes and antibiotic resistance markers (vector therapeutic virus-antibiotic-free)
DNA—Deoxyribonucleic acid
cDNA—Complementary deoxyribonucleic acid
RNA—Ribonucleic acid
mRNA—Messenger ribonucleic acid
bp—base pair
PCR—Polymerase chain reaction
ml—millilitre, μl—microlitre
mm3—cubic millimetre
l—litre
μg—microgram
mg—milligram
g—gram
μM—micromol
mM—millimol
min—minute
s—second
rpm—rotations per minute
nm—nanometre
cm—centimetre
mW—milliwatt
RFU—Relative fluorescence unit
PBS—Phosphate buffered saline

What is claimed is:

1. Gene therapy DNA vector based on gene therapy DNA vector VTvaf17 for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel while the gene therapy DNA vector has the coding region of KRT5 therapeutic gene cloned to gene therapy DNA vector VTvaf17 resulting in a 4929 bp gene therapy DNA vector VTvaf17-KRT5 that has nucleotide sequence SEQ ID No. 1.

2. Gene therapy DNA vector based on gene therapy DNA vector VTvaf17 for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel while the gene therapy DNA vector has the coding region of KRT14 therapeutic gene cloned to gene therapy DNA vector VTvaf17 resulting in a 4575 bp gene therapy DNA vector VTvaf17-KRT14 that has nucleotide sequence SEQ ID No. 2.

3. Gene therapy DNA vector based on gene therapy DNA vector VTvaf17 for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel while the gene therapy DNA vector has the coding region of LAMB3 therapeutic gene cloned to gene therapy DNA vector VTvaf17 resulting in a 6674 bp gene therapy DNA vector VTvaf17-LAMB3 that has nucleotide sequence SEQ ID No. 3.

4. Gene therapy DNA vector based on gene therapy DNA vector VTvaf17 for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel while the gene therapy DNA vector has the coding region of COL7A1 therapeutic gene cloned to gene therapy DNA vector VTvaf17 resulting in a 11990 bp gene therapy DNA vector VTvaf17-COL7A1 that has nucleotide sequence SEQ ID No. 4.

5. Gene therapy DNA vector based on gene therapy DNA vector VTvaf17 carrying KRT5, KRT14, LAMB3, or COL7A1 therapeutic gene as per claim 1, 2, 3, or 4. Said gene therapy DNA vectors are unique due to the fact that each of the constructed gene therapy DNA vectors: VTvaf17-KRT5, or VTvaf17-KRT14, or VTvafl 7-LAMB3, or VTvafl 7-COL7A1 as per claim 1, 2, 3, or 4 due to the limited size of VTvaf17 vector part not exceeding 3200 bp has the ability to efficiently penetrate into human and animal cells and express the KRT5, or KRT14, or LAMB3, or COL7A1 therapeutic gene cloned to it.

6. Gene therapy DNA vector based on gene therapy DNA vector VTvafl 7 carrying KRT5, KRT14, LAMB3, or COL7A1 therapeutic gene as per claim 1, 2, 3, or 4. Said gene therapy DNA vectors are unique due to the fact that each of the constructed gene therapy DNA vectors: VTvaf17-KRT5, or VTvafl 7-KRT14, or VTvafl 7-LAMB3, or VTvafl 7-COL7A1 as per claim 1, 2, 3, or 4 uses nucleotide sequences that are not antibiotic resistance genes, virus genes, or regulatory elements of viral genomes as structure elements, which ensures its safe use for gene therapy in humans and animals.

7. A method of gene therapy DNA vector production based on gene therapy DNA vector VTvafl 7 carrying the KRT5, KRT14, LAMB3, and COL7A1 therapeutic gene as per claim 1, 2, 3, or 4 that involves obtaining each of gene therapy DNA vectors: VTvafl 7-KRT5, or VTvafl 7-KRT 14, or VTvafl 7-LAMB3, or VTvafl 7-COL7A1 as follows: the coding region of the KRT5, or KRT14, or LAMB3, or COL7A1 therapeutic gene as per claim 1, 2, 3, or 4 is cloned to gene therapy DNA vector VTvaf17, and gene therapy DNA vector VTvaf17-KRT5, SEQ ID No. 1, or VTvafl 7-KRT14, SEQ ID No. 2, or VTvafl 7-LAMB3, SEQ ID No. 3, or VTvaf17-COL7A1, SEQ ID No. 4, respectively, is obtained, while the coding region of the KRT5, or KRT14, or LAMB3, or COL7A1 therapeutic gene is obtained by isolating total RNA from the human biological tissue sample followed by the reverse transcription reaction and PCR amplification using the obtained oligonucleotides and cleaving the amplification product by corresponding restriction endonucleases, while cloning to the gene therapy DNA vector VTvaf17 is performed by BamHII and HindIII, or Sail and EcoRI restriction sites, while the selection is performed without antibiotics, at the same time, the following oligonucleotides produced for this purpose are used during gene therapy DNA vector VTvaf17-KRT5, SEQ ID No. 1 production for the reverse transcription reaction and PCR amplification:
KRT5 F
TTTGGATCCACCATGTCTCGCCAGTCAAGTGTGTCCTTC,
KRT5_R
AAT AAGCTT CTAGCTCTT G AAGCT CTTCCGGGAGG,
and the cleaving of amplification product and cloning of the coding region of KRT5 gene to gene therapy DNA vector VTvaf17 is performed by BamHII and HindIII restriction endonucleases,
at the same time, the following oligonucleotides produced for this purpose are used during gene therapy DNA vector VTvaf17-KRT14, SEQ ID No. 2 production for the reverse transcription reaction and PCR amplification:
KRT14_F
TTT GG ATCC ACC AT G ACC ACCT GC AGCCGCC AG,
KRT14 R
AATAAGCTTTCAGTTCTTGGTGCGAAGGACCTGC,
and the cleaving of amplification product and cloning of the coding region of KRT 14 gene to gene therapy DNA vector VTvaf17 is performed by BamHII and HindIII restriction endonucleases, at the same time, the following oligonucleotides produced for this purpose are used during gene therapy DNA vector VTvaf17-LAMB3, SEQ ID No. 3 production for the reverse transcription reaction and PCR amplification:
LAMB3 F
TT AGT CGACC ACC AT GAGACC ATTCTT CCTCTTG,
LAMB3 R
ATAGAATTCACTTGCAGGTGGCATAGTAGAG,
and the cleaving of amplification product and cloning of the coding region of LAMB3 gene to gene therapy DNA vector VTvaf17 is performed by Sail and EcoRI restriction endonucleases, at the same time, the following oligonucleotides produced for this purpose are used during gene therapy DNA vector VTvaf17-COL7A1, SEQ ID No. 4 production for the reverse transcription reaction and PCR amplification:
COL7AI_F
ATCGTCGACCACCATGACGCTGCGGCTTCTGGT,
COL7A1 R
ATAGAATTCAGTCCTGGGCAGTACCTGTC,
and the cleaving of amplification product and cloning of the coding region of COL7A1 gene to gene therapy DNA vector VTvaf17 is performed by Sail and EcoRI restriction endonucleases.

8. A method of use of the gene therapy DNA vector based on gene therapy DNA vector VTvaf17 carrying KRT5, KRT14, LAMB3, and COL7A1 therapeutic gene as per claim 1, 2, 3, or 4 for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel that involves transfection of the cells of patient or animal organs and tissues with the selected gene therapy DNA vector carrying the therapeutic gene based on gene therapy DNA vector VTvaf17, or several selected gene therapy DNA vectors carrying therapeutic genes based on gene therapy DNA vector VTvaf17, from the group of constructed gene therapy DNA vectors carrying therapeutic genes based on gene therapy DNA vector VTvaf17 and/or injection of autologous cells of said patient or animal transfected by the selected gene therapy DNA vector carrying therapeutic gene based on gene therapy DNA vector VTvaf17 or several selected gene therapy DNA vectors carrying the therapeutic genes based on gene therapy DNA vector VTvaf17 from the constructed gene therapy DNA vectors carrying therapeutic genes based on gene therapy DNA vector VTvaf17 into the organs and tissues of the same patient or animal and/or the injection of the selected gene therapy DNA vector carrying therapeutic gene based on gene therapy DNA vector VTvaf17 or several selected gene therapy DNA vectors carrying therapeutic genes based on gene therapy DNA vector VTvaf17 from the group of constructed gene therapy DNA vectors carrying therapeutic genes based on gene therapy DNA vector VTvaf17 into the organs and tissues of the same patient or animal, or the combination of the indicated methods.

9. A method of production of strain for construction of a gene therapy DNA vector as per claim 1, 2, 3, or 4 for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel that involves making electrocompetent cells of Escherichia coli strain SCSI 10-AF and subjecting these cells to electroporation with gene therapy DNA vector VTvaf17-KRT5, or gene therapy DNA vector VTvaf17-KRT14, or gene therapy DNA vector VTvaf17-LAMB3, or gene therapy DNA vector VTvaf17-COL7A1. After that, the cells are poured into agar plates (Petri dishes) with a selective medium containing yeastrel, peptone, 6% sucrose, and 10 pg/ml of chloramphenicol, and as a result, Escherichia coli strain SCSI 10-AF/VTvaf17-KRT5 or Escherichia coli strain SCSI 10-AF/VT vaf 17-KRT 14, or Escherichia coli strain SCSI 10-AF/VTvaf17-LAMB3, or Escherichia coli strain SCSI 10-AF/VTvaf17-COL7A1 is obtained.

10. Escherichia coli strain SCSI 10-AF/VTvaf17-KRT5 obtained as per claim 9 carrying the gene therapy DNA vector VTvaf17-KRT5 for production thereof allowing for antibiotic-free selection during the production of the gene therapy DNA vector for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel.

11. Escherichia coli strain SCSI 10-AF/VTvaf17-KRT14 obtained as per claim 9 carrying the gene therapy DNA vector VTvaf17-KRT14 for production thereof allowing for antibiotic-free selection during the production of the gene therapy DNA vector for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel.

12. Escherichia coli strain SCSI 10-AF/VTvaf17-LAMB3 obtained as per claim 9 carrying the gene therapy DNA vector VTvaf17-LAMB3 for production thereof allowing for antibiotic-free selection during the production of the gene therapy DNA vector for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel.

13. Escherichia coli strain SCSI 10-AF/VTvaf17-COL7A1 obtained as per claim 9 carrying the gene therapy DNA vector VTvaf17-COL7A1 for production thereof allowing for antibiotic-free selection during the production of the gene therapy DNA vector for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel.

14. A method of production on an industrial scale of gene therapy DNA vector based on gene therapy DNA vector VTvaf17 carrying the KRT5, or KRT14, or LAMB3, or COL7A1 therapeutic gene as per claim 1, 2, 3, or 4 for treatment of diseases associated with the disorders of skin, hair, and nails structural organisation, disorder of keratinocyte attachment and connection of epidermis to the sublayers, disorders of wound healing, connective tissues pathologies, including epidermolysis bullosa, Dowling-Degos disease, Oberst-Lehn-Hauss pigmented dermatopathy, Naegeli-Franceschetti-Jadassohn syndrome, and brown enamel that involves production of gene therapy DNA vector VTvaf17-KRT5, or gene therapy DNA vector VTvaf17-KRT14, or gene therapy DNA vector VTvaf17-LAMB3, or gene therapy DNA vector VTvaf17-COL7A1 by inoculating a culture flask containing the prepared medium with seed culture selected from Escherichia coli strain SCSI 10-AF/VTvaf17-KRT5, or Escherichia coli strain SCSI 10-AF/VTvaf17-KRT14, or Escherichia coli strain SCSI 10-AF/VTvaf17-LAMB3, or Escherichia coli strain SCSI 10-AF/VTvaf17-COL7AI, then the cell culture is incubated in an incubator shaker and transferred to an industrial fermenter, then grown to a stationary phase, then the fraction containing the target DNA product is extracted, multi-stage filtered, and purified by chromatographic methods.
